SEQUENCE LISTING (31 sequences)

SEQ ID NO: 1 - DNA, 1296 bp, artificial sequence
  nucleotide sequence encoding Aflibercept
agtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtcgttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 2 - DNA, 1300 bp, artificial sequence
  nucleotide sequence encoding Aflibercept
cagcgacaccggcagacccttcgtggaaatgtacagcgagatccccgagatcatccacatgaccgagggccgcgagctggtgatcccttgcagagtgaccagccccaacatcaccgtgacactgaagaagttccctctggacacactgatccccgacggcaagaggatcatctgggacagcagaaagggcttcatcatcagcaacgccacatacaaagagatcggactgctgacatgcgaggccaccgtgaacggccatctgtacaagaccaactatctgacccaccgccagaccaacaccatcatcgacgtggtgctgagccccagccacggcatcgagctgagcgtgggcgagaagctggtgctgaactgcaccgccagaaccgagctgaatgtgggcatcgacttcaactgggagtaccccagctccaagcaccagcacaagaaactggtgaaccgggatctgaaaacccagagcggcagcgagatgaagaagtttctgagcacactgaccatcgacggcgtgaccagaagcgaccaaggactgtacacatgcgccgccagcagcggactgatgaccaagaagaacagcacattcgtccgggtgcacgagaaggacaagacccacacatgcccaccatgcccagccccagagctgctgggaggcccctccgtgtttctgttccctccaaagcccaaggacactctgatgatcagcagaacccccgaagtgacatgcgtggtggtggacgtgtcccacgaggacccagaagtgaagttcaattggtacgtggacggcgtggaagtgcacaacgccaagaccaagcccagagaggaacagtacaacagcacatacagagtggtgtccgtgctgaccgtgctgcaccaagactggctgaacggcaaagagtacaagtgcaaagtctccaacaaggctctgccagcccccatcgaaaagaccatcagcaaggccaagggccagcctcgcgagccccaagtgtacacactgcctccaagccgggacgagctgaccaagaatcaagtgtctctgacatgtctggtgaaaggcttctaccccagcgatatcgccgtggaatgggagagcaacggccagcccgagaacaactacaagaccacccctcccgtgctggacagcgacggcagcttctttctgtactccaaactgaccgtggacaagagcagatggcagcaaggcaacgtgttcagctgcagcgtgatgcacgaggctctgcacaaccactacacccagaagtctctgtctctgagccccggcaagtga

SEQ ID NO: 3 - protein, 432 aa, artificial sequence
  peptide sequence of Aflibercept
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 23 aa, artificial sequence
  peptide sequence of the TPA signal peptide
MDAMKRGLCCVLLLCGAVFVSPS

SEQ ID NO: 5 - DNA, 68 bp, artificial sequence
  nucleotide sequence encoding the TPA signal peptide
atggatgcaatgaagagagggctctgctgtgtgctgctgctgtgtggagcagtcttcgtttcgcccag

SEQ ID NO: 6 - DNA, 1032 bp, artificial sequence
  Nucleotide sequence encoding Decorin
ggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaa

SEQ ID NO: 7 - DNA, 1035 bp, artificial sequence
  Nucleotide sequence encoding Decorin
ggaccgtttcaacagagaggcttatttgactttatgctagaagatgaggccagcggcatcggccccgaagtgcccgatgatagagatttcgagccctctctgggccccgtgtgtcctttcagatgccagtgtcatctgagagtggtgcagtgcagcgatctgggcctcgacaaagtgcctaaggatctgcctccagacaccacactgctggatctgcagaacaacaagatcaccgagatcaaggacggcgactttaagaatctgaagaatctccacgctctgatcctcgtgaacaacaaaatctccaaagtgtctcccggcgctttcacccctctggtcaagctggaacggctgtatctgagcaagaaccagctgaaagaactgcccgagaagatgcccaagacactgcaagagctgagagcccacgagaacgagatcaccaaagtgcggaaagtgacattcaacgggctgaaccagatgatcgtgatcgagctgggcaccaatcctctgaagtcctccggaatcgagaacggcgccttccaaggcatgaagaagctgagctacatccggatcgccgacaccaacatcaccagcattcctcaagggctgcctccatctctgaccgagctgcatctggacggcaacaagatttccagagtggacgccgcctctctgaagggactgaacaatctggccaaactgggactgagcttcaacagcatcagcgccgtggacaacggctctctggccaacacaccacatctgcgggaactccatctggataacaacaagctgaccagagttcccggcggactggccgagcacaagtacatccaagtggtgtatctccacaacaacaatatcagcgtcgtgggcagcagcgatttctgccctccgggacacaataccaagaaggccagctacagcggagtgtctctgttcagcaatcccgtgcagtactgggagatccagcctagcacattcagatgcgtgtacgtgcggagcgccatccagctgggcaactacaagtgatga

SEQ ID NO: 8 - protein, 343 aa, artificial sequence
  Peptide sequence of Decorin
GPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 9 - DNA, 1032 bp, artificial sequence
  nucleotide sequence encoding Decorin
ggaccgtttcaacagagaggcttatttgactttatgctagaagatgaggcctctggaatcggacctgaggtgcccgacgacagagacttcgaaccttctctgggccctgtgtgccccttcagatgccagtgtcatctgagagtggtgcagtgcagcgacctgggccttgataaggtgcccaaggacctgcctcctgacaccacactgctggacctgcagaacaacaagatcaccgagatcaaggacggcgacttcaagaacctgaagaatctgcacgccctgatcctggtcaacaacaaaatcagcaaggtgtcccctggcgccttcacacctctggtcaagctggaaagactgtacctgagcaagaaccagctgaaagaactgcccgagaagatgcccaagacactgcaagagctgcgggcccacgagaacgagatcaccaaagtgcggaaagtgaccttcaacggcctgaaccagatgatcgtgatcgagctgggcaccaatcctctgaagtccagcggcattgagaacggcgccttccagggcatgaagaagctgagctacatccggatcgccgacaccaacatcaccagcattcctcagggcctgcctccaagcctgacagagctgcatctggacggcaacaagattagcagagtggacgccgcctctctgaagggcctgaacaatctggccaaactgggcctgagcttcaacagcatcagcgccgtggataacggcagcctggccaacacacctcacctgagggaactgcacctggataacaacaagctgaccagagtgcctggcggactggccgagcacaagtacatccaggtggtgtatctccacaacaacaacatctccgtcgtgggcagcagcgacttctgtcctcctggccacaataccaagaaggccagctactctggcgtgtccctgttcagcaaccccgtgcagtactgggagatccagcctagcacctttagatgcgtgtacgtgcggagcgccatccagctgggcaactacaaatga

SEQ ID NO: 10 - DNA, 1032 bp, artificial sequence
  Nucleotide sequence encoding Decorin
ggaccgtttcaacagagaggcttatttgactttatgctagaagacgaggctagcggaattggacctgaagtgcccgacgaccgcgattttgaaccatcactgggacctgtctgcccctttagatgtcagtgccacctgagggtggtgcagtgttctgacctgggcctggataaggtgccaaaggacctgccccctgataccacactgctggacctgcagaacaataagatcaccgagatcaaggacggcgatttcaagaatctgaagaacctgcacgccctgatcctggtgaacaataagatctctaaggtgagcccaggcgcctttacccccctggtgaagctggagagactgtacctgagcaagaatcagctgaaggagctgcccgagaagatgcctaagacactgcaggagctgcgggcccacgagaacgagatcaccaaggtgagaaaggtgacattcaatggcctgaaccagatgatcgtgatcgagctgggcaccaatcccctgaagagctccggcatcgagaacggcgcctttcagggcatgaagaagctgtcctatatccggatcgccgacaccaatatcacatctatccctcagggcctgccacccagcctgacagagctgcacctggacggcaacaagatcagcagagtggatgccgcctccctgaagggcctgaacaatctggccaagctgggcctgtccttcaactccatctctgccgtggacaatggctctctggccaacacccctcacctgagggagctgcacctggataacaataagctgacacgcgtgccaggcggcctggcagagcacaagtacatccaggtggtgtatctgcacaacaataacatctccgtggtgggctctagcgatttctgccctccaggccacaatacaaagaaggccagctactccggcgtgtccctgttttctaaccctgtgcagtattgggagatccagccctctacttttcggtgcgtctatgtcaggtccgccattcagctggggaactacaaataa

SEQ ID NO: 11 - DNA, 1032 bp, artificial sequence
  Nucleotide sequence encoding Decorin
ggaccgtttcaacagagaggcttatttgactttatgctagaagacgaggccagcggcatcggccccgaggtgcccgacgaccgcgacttcgagcccagcctgggccccgtgtgccccttccgctgccagtgccacctgcgcgtggtgcagtgcagcgacctgggcctggacaaggtgcccaaggacctgccccccgacaccaccctgctggacctgcagaacaacaagatcaccgagatcaaggacggcgacttcaagaacctgaagaacctgcacgccctgatcctggtgaacaacaagatcagcaaggtgagccccggcgccttcacccccctggtgaagctggagcgcctgtacctgagcaagaaccagctgaaggagctgcccgagaagatgcccaagaccctgcaggagctgcgcgcccacgagaacgagatcaccaaggtgcgcaaggtgaccttcaacggcctgaaccagatgatcgtgatcgagctgggcaccaaccccctgaagagcagcggcatcgagaacggcgccttccagggcatgaagaagctgagctacatccgcatcgccgacaccaacatcaccagcatcccccagggcctgccccccagcctgaccgagctgcacctggacggcaacaagatcagccgcgtggacgccgccagcctgaagggcctgaacaacctggccaagctgggcctgagcttcaacagcatcagcgccgtggacaacggcagcctggccaacaccccccacctgcgcgagctgcacctggacaacaacaagctgacccgcgtgcccggcggcctggccgagcacaagtacatccaggtggtgtacctgcacaacaacaacatcagcgtggtgggcagcagcgacttctgcccccccggccacaacaccaagaaggccagctacagcggcgtgagcctgttcagcaaccccgtgcagtactgggagatccagcccagcaccttccgctgcgtgtacgtgcgcagcgccatccagctgggcaactacaagtaa

SEQ ID NO: 12 - DNA, 1032 bp, artificial sequence
  Nucleotide sequence encoding Decorin
ggaccgtttcaacagagaggcttatttgactttatgctagaagatgaggcgagtggcattggacctgaagtacccgatgatagagactttgaaccatcattgggcccagtttgcccttttaggtgtcagtgccacctccgggtagttcaatgcagcgatttgggactcgataaagtaccgaaagacttgccaccggacacaacattgctcgatcttcaaaacaacaagatcactgaaataaaggatggagactttaaaaatctgaagaatttgcacgccctcatcctggtcaacaacaagatcagcaaggtgtcccctggagcattcacgcccctcgtaaagttggaacgcctctacctgtctaagaaccagttgaaagaactgcccgagaagatgcctaaaactctgcaagagcttagagctcatgaaaatgaaattaccaaggttcggaaggtaacctttaacggtcttaaccagatgatagtcattgagttgggcacgaacccattgaaatcttctggcatagaaaacggggctttccaggggatgaaaaaactctcatatatccgcatcgcggataccaacatcacatctatacctcaaggtttgcccccgagtttgaccgagcttcacctggatggcaacaagataagccgggtcgacgctgcctcactcaaagggctcaataatctggcgaaactggggttgagtttcaattcaatatctgctgtcgacaacggctcacttgcgaacacaccccatcttagggaacttcatctggacaacaacaagttgacacgggttcctgggggactcgctgaacataaatatatacaggtcgtttatctccataataataatatcagcgttgtaggctcatctgacttctgccctccaggccataatacaaagaaagcgtcatacagtggcgtcagtttgttctctaacccggttcagtattgggagattcaaccgtccacttttcggtgcgtttacgtgaggagtgcgattcagctgggtaactataagtaa

SEQ ID NO: 13 - protein, 16 aa, artificial sequence
  Peptide sequence of the native signal peptide of Decorin
MKATIILLLLAQVSWA

SEQ ID NO: 14 - DNA, 48 bp, artificial sequence
  Nucleotide sequence encoding the native signal peptide of Decorin
atgaaggccactatcatcctccttctgcttgcacaagtttcctgggct

SEQ ID NO: 15 - DNA, 2040 bp, artificial sequence
  Nucleotide sequence encoding transferrin
gtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctatagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaa

SEQ ID NO: 16 - DNA, 2043 bp, artificial sequence
  Nucleotide sequence encoding transferrin
gtgccagataagacagttcgttggtgcgccgtgtctgagcacgaggccacaaagtgccagagcttccgggaccacatgaagtctgtgatccctagcgacggcccttccgtggcttgtgtgaagaaggccagctatctggactgcatcagagccattgccgccaacgaagccgatgccgttacactggatgccggactggtgtacgatgcctatctggccccaaacaatctgaagcccgtggtcgccgagttctacggctctaaagaggaccctcagacattctactacgccgtggccgtggtcaagaaggacagcggctttcagatgaaccagctgcggggcaagaagtcttgtcacaccggacttggaagaagcgccggctggaatatccccatcggactgctgtactgcgatctgcccgagcctagaaagcctctggaaaaggccgtggccaacttcttctctggctcttgtgccccttgcgccgatggcacagattttccacagctctgtcagctgtgtcccggctgtggctgtagcacactgaaccagtactttggctacagcggcgccttcaagtgtctgaaagatggtgctggcgacgtggccttcgtgaagcacagcacaatcttcgagaatctggccaacaaggccgaccgggatcagtacgaactgctgtgcctcgacaacaccagaaagccagtggacgagtacaaggactgccatctggctcaagtgcctagccacacagtggttgccagatccatgggcggcaaagaggatctgatctgggagctgctgaatcaagcccaagagcacttcggcaaggacaagagcaaagagttccagctgttcagcagccctcacggcaaggatctgctgttcaaggatagcgcccacggatttctgaaagtgcctcctcggatggacgccaagatgtatctgggctacgagtacgtgaccgccatccggaatctgagagaaggcacatgcccagaggctcccaccgatgagtgtaaaccagtgaagtggtgcgctctgtctcaccacgagagactgaagtgtgacgagtggtccgtgaacagcgtgggcaagattgagtgtgtgtccgccgagacaaccgaggactgtatcgccaagatcatgaacggcgaggccgacgctatgtctctggatggcggatttgtgtacattgccggaaagtgtggactggtgccagtgctggccgagaactacaacaagagcgacaactgcgaggataccccagaggccggatattttgccgtggcagtcgtgaagaagtccgccagcgatctgacatgggacaatctcaagggcaagaaaagctgccacaccgccgtgggaagaacagccggatggaacattcctatggggctgctgtacaacaaaatcaaccactgccgcttcgacgagttcttcagcgaaggatgtgctcccggcagcaagaaagacagctctctgtgcaagctgtgcatgggcagcggactgaatctgtgcgagcccaacaacaaagagggctactacggctacaccggggcctttagatgtctggttgagaagggcgacgttgcatttgtgaaacaccagaccgtgcctcagaacaccggcggcaagaatcccgatccttgggccaagaatctgaacgagaaggactatgagctgctctgtctggacggcacccggaaaccagtggaagaatacgccaactgtcatctggcaagagccccaaatcacgccgtcgtgaccagaaaggacaaagaggcttgcgtccacaagattctgcggcagcagcagcatctgttcggcagcaatgtgaccgactgcagcggcaacttctgtctgttcagaagcgagacaaaggatctcctcttccgcgacgataccgtgtgtctcgccaagctgcacgaccggaacacatacgagaagtatctgggagaagagtatgtgaaggctgtgggcaatctgcggaagtgcagcacatcttctctgctcgaggcttgcacatttcggcggccttgatga

SEQ ID NO: 17 - protein, 679 aa, artificial sequence
  Peptide sequence of human transferrin
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 18 - protein, 19 aa, artificial sequence
  Peptide sequence of the native signal peptide of human transferrin
MRLAVGALLVCAVLGLCLA

SEQ ID NO: 19 - DNA, 57 bp, artificial sequence
  Nucleotide sequence encoding the native signal peptide of transferrin
atgaggctcgccgtgggagccctgctggtctgcgccgtcctggggctgtgtctggct

SEQ ID NO: 20 - DNA, 57 bp, artificial sequence
  Nucleotide sequence encoding the signal peptide of transferrin
atgagactggctgtgggagcactgcttgtgtgtgctgttctgggactgtgtctggcc

SEQ ID NO: 21 - DNA, 1249 bp, artificial sequence
  nucleotide sequence encoding an anti-TNF-alpha fusion protein
actggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacactggttccgcgtggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatga

SEQ ID NO: 22 - protein, 415 aa, artificial sequence
  peptide sequence of an anti-TNF-alpha fusion protein
LVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTLVPRGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 29 aa, artificial sequence
  Peptide sequence of a signal peptide
MGLSTVPDLLLPLVLLELLVGIYPSGVIG

SEQ ID NO: 24 - DNA, 86 bp, artificial sequence
  Nucleotide sequence encoding the signal peptide of sequence SEQ ID NO 23
atgggcctctccaccgtgcctgacctgctgctgccgctggtgctcctggagctgttggtgggaatatacccctcaggggttattgg

SEQ ID NO: 25 - DNA, 3642 bp, artificial sequence
  nucleotide sequence encoding complement factor H
gaagattgcaatgaacttcctccaagaagaaatacagaaattctgacaggttcctggtctgaccaaacatatccagaaggcacccaggctatctataaatgccgccctggatatagatctcttggaaatataataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatag

SEQ ID NO: 26 - protein, 1213 aa, artificial sequence
  peptide sequence of complement factor H
EDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 27 - protein, 18 aa, artificial sequence
  Peptide sequence of the native signal peptide of factor H
MRLLAKIICLMLWAICVA

SEQ ID NO: 28 - DNA, 54 bp, artificial sequence
  Nucleotide sequence encoding the native signal peptide of factor H
atgagacttctagcaaagattatttgccttatgttatgggctatttgtgtagca

SEQ ID NO: 29 - protein, 20 aa, artificial sequence
  peptide sequence of the signal peptide of HTLV-1 Env
MGKFLATLILFFQFCPLIFG

SEQ ID NO: 30 - DNA, 60 bp, artificial sequence
  nucleotide sequence encoding the signal peptide of HTLV-1 Env
atgggtaagtttctcgccactttgattttattcttccagttctgccccctcatcttcggt

SEQ ID NO: 31 - DNA, 281 bp, artificial sequence
  sequence of the gamma origin of replication of the plasmid E. coli R6K
gatcagcagttcaacctgttgatagtatgtactaagctctcatgtttaatgtactaagctctcatgtttaatgaactaaaccctcatggctaatgtactaagctctcatggctaatgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaatggttgtggacaacaagcc

(19) United States
(12) Patent Application Publication
(10) Pub. No.: US 2024/0189448 A1
(43) Pub. Date: Jun. 13, 2024

(54) DNA STRUCTURE FOR TREATING OCULAR PATHOLOGIES

(71) Applicant: EYEVENSYS, PARIS (FR)

(72) Inventors: Thierry BORDET, Paris (FR); Elise ORHAN, Rueil-Malmaison (FR); Karine BIGOT, Lunay (FR); Ronald BUGGAGE, Paris (FR)

(21) Appl. No.: 18/003,585

(22) PCT Filed: Jun. 30, 2021
(86) PCT No.: PCT/EP2021/068085

§ 371 Date: Dec. 28, 2022

(30) Foreign Application Priority Data
Jun. 30, 2020 (FR) FR2006898

(51) Int. Cl. A61K 48/00; A61K 38/17; A61P 27/02; C12N 15/62; C12N 15/85

(52) U.S. Cl. CPC A61K 48/005; A61K 38/1709; A61K 38/179; A61K 48/0075; A61P 27/02; C12N 15/62; C12N 15/85; C07K 2319/30

(57) ABSTRACT

The present invention mainly relates to a DNA structure for use in treating an ocular pathology and for the non-viral transfer of nucleic acids into the muscular cells of the eyeball of a patient suffering from the ocular pathology; characterised in that it particularly comprises a first sequence encoding a first therapeutic protein and a second sequence encoding a second therapeutic protein which is different from the first therapeutic protein, the DNA structure being administered to the patient by injection into a ciliary muscle then electrotransfer into the cells of the ciliary muscle.

### TECHNICAL FIELD

The present invention relates to a new DNA construct as well as the application thereof in the treatment of ocular pathologies by nonviral gene therapy. A method according to the invention relates more particularly to said DNA construct making possible the targeted intraocular production of two therapeutic proteins during a period that may last for up to several months. The DNA construct and its use according to the invention are more particularly suitable for treating pathologies of the retina by means of an injection of the DNA construct into the ciliary muscle followed by electrotransfer for long-lasting intraocular production of the therapeutic proteins of interest.

### PRIOR ART

Blindness related to metabolic and inflammatory diseases or to age is increasing considerably and poses a more and more significant social problem in Europe and throughout the world in terms of public health. The main causes of blindness are related to pathologies of the retina, including age-related macular degeneration (ARMD), diabetic retinopathies (DR), uveitis, glaucoma, retinitis pigmentosa, hemorrhages following eye injury and retinal detachment. Age-related macular degeneration (ARMD) leading to blindness has a prevalence of the order of 8.7%, and affects nearly 26% of the population aged 50 years and over. ARMD is becoming a major problem of public and social health on account of the increasing age of the population. The continual major increase in the incidence of diabetes is the major cause of diabetic retinopathies (DR), which in their turn are becoming a priority of public health and scientific research. The statistics show that type 2 diabetes will affect, from now to 2030, more than 4.5% of the population, nearly 30% of whom will suffer diabetic retinopathy. Finally, uveitis represents a group of inflammatory ocular diseases whose prevalence is estimated at 1/1000 and incidence at 0.5/1000. Uveitis is responsible for 10% of cases of blindness and therefore, although rarer than the diseases mentioned above, has a major social and economic impact in young patients of working age. Glaucoma is the second cause of irreversible blindness in the world. The number of people with glaucoma in the world is expected to increase from 76 million in 2020 to 111 million in 2040. Glaucoma is characterized by abnormal intraocular pressure inducing a progressive optical neuropathy characterized by degeneration of the retinal ganglion cells and a loss of visual field. The intraocular pressure is currently the only risk factor for which there are treatments. However, the glaucomatous damage persists in nearly 50% of patients, despite a decrease in intraocular pressure. Retinitis pigmentosa represents a clinically and genetically heterogeneous group of hereditary disorders of the retina characterized by a progressive loss of photoreceptors at the periphery of the retina, which then progresses to the macula. The visual deficiency is generally manifested by night blindness and a progressive loss of visual field. Its prevalence is from 1/3000 to 1/5000. More than 50 genes responsible for retinitis pigmentosa have been identified to date.

For treating certain of these pathologies, intraocular, or even intravitreal, injections of therapeutic agents have been developed. In 2006, the first therapeutic proteins of the anti-VEGF type (VEGF: Vascular Endothelial Growth Factor) were administered by the intraocular route for treating choroid neovascularization in ARMD. We may mention in particular, as an example of a protein of the anti-VEGF type, Lucentis® (ranibizumab), which has been given marketing authorization for treating choroid neovascularization in ARMD and diabetic macular edema. Intraocular injections of therapeutic proteins, and in particular of recombinant proteins, have since become common for treating macular edemas in ARMD, diabetic retinopathy and venous occlusions.

In order to ensure a continuing effect on the ocular pathologies, the anti-VEGFs described above are administered once a month or at best once every two months depending on the patients. It is therefore necessary to monitor each patient in order to determine the frequency of administration of the anti-VEGFs so that the treatment is fully effective. This monitoring generates considerable stress for the patients, careers and nursing staff, which most often results in treatment that is suboptimal and ineffective in the long term (Ciulla 2020, Ophthalmology Retina 2020; 4: 19-30). Moreover, this method of treatment induces changes in the level of therapeutic protein in the patient's ocular sphere, namely a high concentration at the time of injection (a peak) and a concentration that gradually diminishes and tends toward zero until the next injection. Therefore the concentration of therapeutic protein is irregular and is not optimal for the entire duration of the treatment. Moreover, the risk of side effects associated with intravitreal administration increases with repeated administration (Schargus 2020, Clinical Ophthalmology 2020: 14 897-904).

Among the ocular pathologies, uveitis is defined as an inflammatory process that affects the iris, ciliary body or the choroid of the patient's eye, these three elements forming the uvea. It is a general term that covers several different pathologies whose causes remain unknown but are generally of two types: infectious uveitides and noninfectious uveitides. A distinction is made between anterior uveitis, which affects the iris or the ciliary body and is the commonest of the uveitides in countries in the west; intermediate uveitis, which affects the anterior vitreous body; and posterior uveitis, which affects the choroid and the retina. Noninfectious uveitis often has an autoimmune component. Acute anterior uveitis associated with the HLA-B27 antigen represents the primary cause of uveitis (Rothova et al., Br J Ophthalmol. 1992; 76:137-41). Moreover, anterior uveitis is found associated with many rheumatologic diseases such as sarcoidosis. Posterior uveitis of noninfectious cause is most often associated with Behçet disease, Vogt Koyanagi Harada disease, birdshot chorioretinopathy, etc.

Treatment with corticoids by the oral and/or topical route is widely used in the treatment of noninfectious posterior uveitis. Moreover, for the most refractory pathologies, immunosuppressants may be added to the treatment to increase the anti-inflammatory effects of the corticosteroids. This relates in particular but not exclusively to ciclosporin, methotrexate, azathioprine, mycophenolate mofetil, tacrolimus and chlorambucil. For about the last ten years, treatments using anti-TNF alpha antibodies could also be used, including Humira® (Adalimumab), recently approved for treating noninfectious uveitis with inflammation of the back of the eye.

Clinical trials into the use of the following therapeutic compounds: ciclosporin A, rapamycin, tacrolimus, and anti-TNF alpha, have been conducted to evaluate the efficacy and safety of these compounds and thus be able to treat autoimmune ocular pathologies such as Behçet disease. However, it has been shown that systemic administration of these therapeutic compounds leads in the long term to many side effects and that for some of them, their local administration provides little efficacy or poor tolerance by the patient.

The methods of treatment described above therefore have several drawbacks. The therapeutic compounds administered by the systemic and topical route lead to numerous side effects. Recombinant proteins administered by intravitreal injections must be administered frequently, with large fluctuations in concentrations between each administration. Repetition of these injections is still extremely onerous and stressful for the patients and may also cause effects (increase in intraocular pressure, intraocular inflammation, endophthalmitis, cataract, etc.). Thus, in the end many patients abandon their treatment.

In order to overcome this problem, the inventors have previously proposed, as illustrated in particular in application FR3031112, to reduce the number and/or frequency of surgical interventions, and therefore reduce the invasive aspect of the intraocular injections, while ensuring a stable and constant production of a therapeutic protein for several months through application of a DNA construct intended for nonviral transfer of nucleic acids into the muscle cells of a patient's ocular sphere. This construct comprises an origin of replication, a promoter allowing expression of the DNA in the patient's ocular sphere, one or more sequences promoting the expression of DNA in the patient's ocular sphere, and a polynucleotide coding for a therapeutic protein selected for its activity in the treatment of ocular pathologies, said construct being delivered into the ocular sphere by direct injection into the ciliary muscle followed by electrotransfer.

Treatment of the ocular pathologies mentioned above may, however, require the application of two active therapeutic ingredients, of a second compound for potentiating the efficacy of an active therapeutic ingredient, or of a compound consisting of 2 peptide subunits.

In the context of the method mentioned above, it could thus be envisaged to administer to the patient a composition comprising two types of DNA constructs, differing in that a first type allows expression of the first molecule of interest while a second type allows expression of the second molecule of interest. Such a method would not, however, be ideal, (i) in that it would require the development of two products, which would lead to an increase in the costs of development and production, and to the need to evaluate the activity and safety of each of the products taken separately and in combination, (ii) in that it would impose a considerable constraint in terms of dosage, halving the maximum dose of each of the two active therapeutic ingredients that may be administered, and finally (iii) in that it would not allow complete control of the quantity of each of these constructs penetrating the targeted cells, which would lead in consequence to uncertainty regarding the proportions of these molecules of interest expressed at the level of the eye. Such constraints and uncertainties are undesirable in the context of the treatment of a pathology.

The inventors consequently propose, in the context of the present invention, to employ a DNA construct comprising the sequences coding for the two proteins of interest.

### SUMMARY OF THE INVENTION

The present invention therefore relates firstly to a DNA construct for use thereof in the treatment of an ocular pathology,
said DNA construct being intended for the nonviral transfer of nucleic acids into the muscle cells of the ocular sphere of a patient with said ocular pathology;
said DNA construct being characterized in that it comprises:
(a) a bacterial or prokaryotic origin of replication, in particular bacterial,
(b) one or more sequences promoting the expression of DNA in the patient's ocular sphere,
(c) a first nucleotide sequence coding for:
   a first therapeutic protein, and
   a signal peptide allowing secretion of this first therapeutic protein,
this signal peptide being contiguous with the sequence of the first therapeutic protein, at the N-terminal of said first therapeutic protein;
(d) a promoter allowing expression of this first therapeutic protein in the patient's ocular sphere;
(e) a polyadenylation sequence at 3′ of the first nucleotide sequence;
(f) a second nucleotide sequence coding for:
   a second therapeutic protein, different than the first therapeutic protein, and
   a signal peptide allowing secretion of this second therapeutic protein,
the signal peptide being contiguous with the sequence of the second therapeutic protein, at the N-terminal of said second therapeutic protein;
(g) a promoter allowing expression of this second therapeutic protein in the patient's ocular sphere; and
(h) a polyadenylation sequence at 3′ of the second nucleotide sequence;
said DNA construct being administered to the patient by injection into a ciliary muscle and then electrotransfer into the cells of the ciliary muscle.

In fact, against all expectations, the significant increase in the size of the DNA construct, a consequence of introducing not one but two sequences coding for the molecules of interest, does not have a negative effect on its capacity to penetrate the targeted cells in the context of a method such as indicated above, comprising not only direct injection of said construct into the ciliary muscle, but also a step of electrotransfer.

Consequently, a method and a construct according to the invention allow advantageously, and against all expectations:
not only to increase the possibilities of treatments of ocular pathologies, by allowing the production in situ of two active ingredients or of one active ingredient and a suitable compound for potentiating the activity/efficacy of the active ingredient;
but also to maintain, without decreasing them, the advantages of the method indicated above in terms of reduced number/frequency of surgical interventions, and consequently of reduction of the invasive aspect of the intraocular injections, while ensuring stable, continual production of the therapeutic proteins for several months.

According to one embodiment, the first therapeutic protein of a DNA construct according to the invention is a protein of the anti-VEGF type, in particular selected from the group consisting of S-Flt1, aflibercept, conbercept, brolucizumab, and in particular of a protein having at least 85% sequence identity with the peptide sequence SEQ ID NO: 3, this protein more particularly being aflibercept.

According to one embodiment, the first therapeutic protein is encoded by a nucleotide sequence having at least 75% sequence identity with the sequence SEQ ID NO: 1, and is more particularly encoded by the nucleotide sequence SEQ ID NO: 2.

According to one embodiment, the second therapeutic protein of a DNA construct according to the invention is a protein having at least 85% sequence identity with the sequence SEQ ID NO: 8, this protein more particularly being decorin.

According to one embodiment, the second therapeutic protein is encoded by a nucleotide sequence having at least 70% sequence identity with the sequence SEQ ID NO: 6, and in particular by a sequence selected from the group consisting of the nucleotide sequences SEQ ID NO: 7, SEQ ID NO: 9, SEQ ID NO: 10, SEQ ID NO: 11 and SEQ ID NO: 12, more particularly consisting of the sequence SEQ ID NO: 7 and the sequence SEQ ID NO: 1, and in particular the sequence SEQ ID NO: 11.

According to one embodiment, a DNA construct for use thereof according to the invention is such that:
(c) the first nucleotide sequence codes for:
   a protein having at least 85% sequence identity with the sequence SEQ ID NO: 3, this protein more particularly being aflibercept; and
   a signal peptide of peptide sequence SEQ ID NO: 4; and
(f) the second nucleotide sequence codes for:
   a protein having at least 85% sequence identity with the sequence SEQ ID NO: 8, this protein more particularly being decorin; and
   a signal peptide of peptide sequence SEQ ID NO: 13.

According to one embodiment, the origin of replication of a DNA construct such as mentioned above is bacterial, and is in particular an origin of replication derived from the natural plasmid R6K of Escherichia coli, in particular the origin of replication R6K gamma of the natural plasmid R6k of Escherichia coli, in particular of sequence SEQ ID NO: 31.

The DNA construct according to the invention may be of linear or circular shape, in particular of circular shape. In a particular embodiment, the DNA construct according to the invention is a circular plasmid.

In a particular embodiment, the DNA construct is a naked DNA construct.

According to one embodiment, a DNA construct for use thereof as mentioned above is characterized in that the ocular pathology is a retinal degeneration, in particular a retinal degeneration selected from the group consisting of wet or dry age-related macular degeneration (ARMD); diabetic retinopathies (DR); a retinal venous occlusion, in particular a central retinal vein occlusion (CRVO) or a branch retinal vein occlusion (BRVO); a myopic choroid neovascularization (CNV); a uveitis, in particular a noninfectious uveitis; a retinitis pigmentosa and a glaucoma, and more particularly in that the retinal degeneration is selected from the group consisting of age-related macular degeneration (ARMD), in particular the (wet) neovascular form of ARMD; a decline of visual acuity due to diabetic macular edema (DME); a retinal venous occlusion, in particular a central retinal vein occlusion (CRVO) or a branch retinal vein occlusion (BRVO); and a myopic choroid neovascularization (CNV).

The present invention further relates to a DNA construct intended for the nonviral transfer of nucleic acids into the muscle cells of a patient's ocular sphere for treating ocular pathologies, characterized in that it comprises:
(a) a bacterial or prokaryotic origin of replication, in particular bacterial,
(b) one or more sequences promoting the expression of DNA in the patient's ocular sphere,
(c) a first nucleotide sequence coding for:
   a first therapeutic protein, said first therapeutic protein being a protein having at least 85% sequence identity with the sequence SEQ ID NO: 3, this protein more particularly being aflibercept, and
   a signal peptide allowing secretion of this first therapeutic protein, in particular a signal peptide of peptide sequence SEQ ID NO: 4,
this signal peptide being contiguous with the sequence of the first therapeutic protein, at the N-terminal of said first therapeutic protein;
(d) a promoter allowing expression of this first therapeutic protein in the patient's ocular sphere;
(c) a polyadenylation sequence at 3′ of the first nucleotide sequence;
(f) a second nucleotide sequence coding for:
   a second therapeutic protein, said second therapeutic protein being a protein having at least 85% sequence identity with the sequence SEQ ID NO: 8, this protein more particularly being decorin, and
   a signal peptide allowing secretion of this first therapeutic protein, in particular a signal peptide of peptide sequence SEQ ID NO: 13,
this signal peptide being contiguous with the sequence of the second therapeutic protein, at the N-terminal of said second therapeutic protein;
(g) a promoter allowing expression of this second therapeutic protein in the patient's ocular sphere; and
(h) a polyadenylation sequence at 3′ of the second nucleotide sequence.

In particular, the DNA construct according to the invention is such that:
c) the first nucleotide sequence comprises:
   a nucleotide sequence coding for aflibercept, and in particular a sequence having at least 75% sequence identity with the sequence SEQ ID NO: 1, and is in particular the sequence SEQ ID NO: 2; and
   the nucleotide sequence SEQ ID NO: 5 coding for a signal peptide; and
(f) the second nucleotide sequence comprises:
   a nucleotide sequence coding for decorin, more particularly a nucleotide sequence having at least 70% sequence identity with the sequence SEQ ID NO: 6, more particularly a sequence selected from the group consisting of the sequences SEQ ID NO: 7, SEQ ID NO: 9, SEQ ID NO: 10, SEQ ID NO: 11 and SEQ ID NO: 12, and is in particular the sequence SEQ ID NO: 7 or the sequence SEQ ID NO: 11; and
   the nucleotide sequence SEQ ID NO: 14 coding for a signal peptide.

### BRIEF DESCRIPTION OF THE DRAWINGS

FIG. 1 shows a DNA construct according to the invention (Plasmid A).

FIG. 2 shows a DNA construct not according to the invention as it only codes for a single therapeutic protein (transferrin—same sequence as that used in Plasmid A), the latter being, just as in Plasmid A, under the control of a promoter of the CMV type (Plasmid a′).

FIG. 3 shows the variation of the concentration of transferrin of sequence SEQ ID NO: 17 (ordinate: pg/mL) in the ocular fluids of rats from 3 to 30 days (abscissa: days post-electrotransfer (D0)) after administration of a construct according to the invention (Plasmid A) or of a construct not according to the invention (Plasmid a′) as it only codes for transferrin, the latter being under the control of the same promoter as in plasmid A, in the ciliary muscle of both eyes of said rats. Each group of rats has thus been administered a specific construct from the two mentioned.

FIG. 4 shows the variation of the concentration of anti-TNF-alpha fusion protein of sequence SEQ ID NO: 22 (ordinate: pg/mL) in the ocular fluids of rats from 3 to 30 days (abscissa: days post-electrotransfer (D0)) after administration of a construct according to the invention (Plasmid A) in the ciliary muscle of both eyes of said rats.

FIG. 5 shows a DNA construct according to the invention (Plasmid B).

FIG. 6 shows a DNA construct not according to the invention as it only codes for a single therapeutic protein (aflibercept—same sequence as that used in Plasmid B, i.e. the sequence SEQ ID NO: 2), the latter being, just as in Plasmid B, under the control of a promoter of the CAG type (Plasmid b′).

FIG. 7 shows the variation of the concentration of aflibercept of sequence SEQ ID NO: 3 (ordinate: pg/mL) in the ocular fluids of rats from 3 to 21 days (abscissa: days post-electrotransfer (D0)) after administration of a construct according to the invention (Plasmid B) or of a construct not according to the invention (Plasmid b′) as it only codes for aflibercept, the latter being under the control of the same promoter as in plasmid B, in the ciliary muscle of both eyes of said rats. Each group of rats was thus administered a specific construct from the two mentioned.

FIG. 8 shows the variation of the concentration of decorin of sequence SEQ ID NO: 8 (ordinate: pg/mL) in the ocular fluids of rats from 3 to 21 days (abscissa: days post-electrotransfer (D0)) after administration of a construct according to the invention (Plasmid B) in the ciliary muscle of both eyes of said rats.

FIG. 9 shows a DNA construct according to the invention (Plasmid C).

FIG. 10 shows the percentage of effect giving severe leakage (grade 3) (ordinate: % Grade 3 CNV Lesions) as a function of the treatments (abscissa). On the left: vehicle (control). On the right: Plasmid C.

### DETAILED DESCRIPTION

### Definitions

In the context of the present text, the terms “treat” and “treatment” associated with an ocular pathology denote a decrease, or even an interruption of said pathology.

The term “patient” as used in the present text preferably denotes a mammal, including a nonhuman mammal, and more particularly a human being.

The terms “first nucleotide sequence” and “second nucleotide sequence” are used in the present text in order to allow, during reading of the latter, to make a clear distinction between these two sequences and the proteins that they encode.

Said nucleotide sequences correspond to expression cassettes, each of these cassettes being as defined hereunder.

These terms “first nucleotide sequence” and “second nucleotide sequence” do not, however, aim to indicate the order in which these sequences/expression cassettes are present in a construct according to the invention. Thus, according to one embodiment, in the sense of reading a construct according to the invention, the first nucleotide sequence may be present before the second nucleotide sequence. In another embodiment, in the sense of reading a construct according to the invention, the second nucleotide sequence may be present before the first nucleotide sequence.

In accordance with what is indicated above, the “first nucleotide sequence” comprises a sequence coding for a first therapeutic protein and a sequence coding for a signal peptide, these sequences being, within the “first nucleotide sequence”, in the order as specifically indicated relative to one another, namely that the sequence coding for the signal peptide is such that the signal peptide is at N-terminal of the first therapeutic protein, i.e. the sequence coding for the signal peptide is at 5′ of the sequence coding for the first therapeutic protein. Moreover, in accordance with what is indicated above, the “second nucleotide sequence” comprises a sequence coding for a second therapeutic protein and a sequence coding for a signal peptide, these sequences being, within the “second nucleotide sequence”, in the order as specifically indicated relative to one another, namely that the sequence coding for the signal peptide is such that the signal peptide is at N-terminal of the second therapeutic protein, i.e. the sequence coding for the signal peptide is at 5′ of the sequence coding for the second therapeutic protein.

The “percentage identity” between two amino acid or nucleic acid sequences, in the sense of the present invention, is determined by comparing the two optimally aligned sequences, through a comparison window.

The part of the nucleotide sequence in the comparison window may thus comprise additions or deletions (for example “gaps”) relative to the reference sequence (which does not comprise these additions or these deletions) so as to obtain an optimal alignment between the two sequences.

The percentage identity is calculated by determining the number of positions at which an identical amino acid (or an identical nucleic base) is observed for the two sequences compared, then dividing the number of positions at which there is identity between the two amino acids (or between the two nucleic bases) by the total number of positions in the comparison window, and then multiplying the percentage result in order to obtain the percentage of amino acid identity (or nucleotide identity) of the two sequences between them.

The optimal alignment of the sequences for comparison may be carried out by computer using known algorithms.

Totally preferably, the percentage sequence identity is determined using the CLUSTAL W software (version 1.82), the parameters being fixed as follows: (1) CPU MODE=ClustalW mp; (2) ALIGNMENT=“full”; (3) OUTPUT FORMAT=“aln w/numbers”; (4) OUTPUT ORDER=“aligned”; (5) COLOR ALIGNMENT=“no”; (6) KTUP (word size)=“default”; (7) WINDOW LENGTH=“default”; (8) SCORE TYPE=“percent”; (9) TOPDIAG=“default”; (10) PAIRGAP=“default”; (11) PHYLOGENETIC TREE/TREE TYPE=“none”; (12) MATRIX=“default”; (13) GAP OPEN=“default”; (14) END GAPS=“default”; (15) GAP EXTENSION=“default”; (16) GAP DISTANCES=“default”; (17) TREE TYPE=“cladogram” and (18) TREE GAP DISTANCES=“hide”.

In the sense of the invention, an amino acid sequence having for example at least 80% amino acid identity with a reference amino acid sequence includes the amino acid sequences having at least 81%, 82%, 83%, 84%, 85%, 86%, 87%, 88%, 89%, 90%, 91%, 92%, 93%, 94%, 95%, 96%, 97%, 98% or at least 99% amino acid identity with said reference sequence.

In the sense of the invention, a nucleotide sequence having for example at least 80% nucleotide identity with a reference nucleotide sequence includes the nucleotide sequences having at least 81%, 82%, 83%, 84%, 85%, 86%, 87%, 88%, 89%, 90%, 91%, 92%, 93%, 94%, 95%, 96%, 97%, 98% or at least 99% nucleotide identity with said reference sequence.

### DNA Construct

As mentioned above, the present invention relates firstly to a DNA construct for use thereof in the treatment of an ocular pathology.

This construct is intended for the nonviral transfer of nucleic acids into the muscle cells of the ocular sphere of a patient with said ocular pathology.

Moreover, a DNA construct according to the present invention is characterized in that it comprises (a) a bacterial or prokaryotic origin of replication.

According to a particular embodiment, said origin of replication is in particular bacterial and may for example be an origin of replication of the Escherichia coli type, more particularly selected from the group consisting of an origin of replication derived from the natural plasmid R6K of Escherichia coli, in particular the origin of replication R6K gamma of the natural plasmid R6k of Escherichia coli; and the origin of replication pUC OriC.

The origins of replication derived from the natural plasmid R6K of Escherichia coli are in particular defined in patent EP1366176B2.

Moreover, a DNA construct according to the invention is characterized in that it comprises (b) one or more sequences promoting expression of the DNA in the patient's ocular sphere. Such sequences promoting the expression of DNA are familiar to a person skilled in the art, such as for example the sequences of the enhancer type, also called amplifier or activator sequences. We may mention for example the enhancer sequences derived from cytomegalovirus (CMV) and/or the tumoral virus with simian DNA SV40.

Moreover, a DNA construct according to the invention is also characterized in that it comprises (c) a first nucleotide sequence coding in particular for a first therapeutic protein as well as (f) a second nucleotide sequence coding in particular for a second therapeutic protein, different than the first therapeutic protein.

According to a particular embodiment, a DNA construct according to the invention only comprises two coding sequences for therapeutic proteins, i.e. only comprises two expression cassettes, each of these expression cassettes comprising one of the two coding sequences for a therapeutic protein.

These first and second therapeutic proteins may in particular be selected from proteins known for their effect on ocular pathologies.

The effects of these two proteins may be additional or complementary. Moreover, one of these two proteins may have a potentiating effect on the therapeutic activity of the other protein produced starting from the DNA construct according to the invention.

In particular, the first and the second therapeutic proteins, different than one another, may for example be selected from the group consisting of:
(i) a protein having at least 85% sequence identity with the sequence SEQ ID NO: 17, this protein more particularly being transferrin;
(ii) a protein having antifibrotic properties, such as the protein BMP7 (Bone Morphogenic Protein 7), a protein of the anti TGF-beta type, a protein of the anti FGF2 type, a protein of the anti CTGF type (connective tissue growth factor), and in particular a protein having at least 85% sequence identity with the sequence SEQ ID NO: 8, this protein more particularly being decorin;
(iii) a protein having anti-inflammatory properties, in particular a protein of the anti-INF type, such as for example hTNFR-Is, hTNFR-Is/mlgG1, Lenercept or the fusion protein comprising the extracellular domain of the human receptor p55 to TNF alpha coupled via a hinge to the constant fragment of human immunoglobulin IgG1, more particularly a protein of the anti TNF-alpha type, in particular a fusion protein comprising the extracellular domain of the human receptor p55 to INF alpha coupled via a hinge to the constant fragment of human immunoglobulin IgG1 (Peppel et al., J Exp Med, 174: 1483-1489—Murphy et al., Arch Ophthalmol, 22: 845-851), and more particularly a protein having at least 85% sequence identity with the sequence SEQ ID NO: 22;
(iv) a protein of the anti-VEGF type, in particular selected from the group consisting of S-Flt1, aflibercept, conbercept, brolucizumab, and in particular a protein having at least 85% sequence identity with the peptide sequence SEQ ID NO: 3, this protein more particularly being aflibercopt;
(v) a protein having antiangiogenic properties, such as angiostatin, endostatin, thrombospondin, a protein of the anti angiopoietin-2 type, a protein of the anti FGF2 type, a protein of the anti PLGF type, a protein of the anti PDGF type; and
(vi) a protein regulating the activation of complement, such as complement factor I (CFI), and a protein having at least 85% sequence identity with the sequence SEQ ID NO: 26, this protein more particularly being complement factor H.

In particular, (i) a protein having at least 85% sequence identity with the sequence SEQ ID NO: 17 comprises a protein having at least 86%, at least 87%, at least 88%, at least 89%, at least 90%, at least 91%, at least 92%, at least 93%, at least 93%, at least 94%, at least 95%, at least 96%, at least 97%, at least 98%, at least 99% and 100% sequence identity with the sequence SEQ ID NO: 17. In particular, this protein is more particularly transferrin (i.e. a protein having 100% sequence identity with the sequence SEQ ID NO: 17).

In particular, (ii) a protein having at least 85% sequence identity with the sequence SEQ ID NO: 8 comprises a protein having at least 86%, at least 87%, at least 88%, at least 89%, at least 90%, at least 91%, at least 92%, at least 93%, at least 93%, at least 94%, at least 95%, at least 96%, at least 97%, at least 98%, at least 99% and 100% sequence identity with the sequence SEQ ID NO: 8. In particular, this protein is more particularly decorin (i.e. a protein having 100% sequence identity with the sequence SEQ ID NO: 8).

In particular, (iii) a protein having at least 85% sequence identity with the fusion protein of sequence SEQ ID NO: 22 comprises a protein having at least 86%, at least 87%, at least 88%, at least 89%, at least 90%, at least 91%, at least 92%, at least 93%, at least 93%, at least 94%, at least 95%, at least 96%, at least 97%, at least 98%, at least 99% and 100% sequence identity with the sequence SEQ ID NO: 22. In particular, this protein is more particularly the fusion protein comprising the extracellular domain of the human receptor p55 to TNF alpha coupled via a hinge to the constant fragment of human immunoglobulin IgG1 of sequence SEQ ID NO: 22.

In particular, (iv) a protein having at least 85% sequence identity with the sequence SEQ ID NO: 3 comprises a protein having at least 86%, at least 87%, at least 88%, at least 89%, at least 90%, at least 91%, at least 92%, at least 93%, at least 93%, at least 94%, at least 95%, at least 96%, at least 97%, at least 98%, at least 99% and 100% sequence identity with the sequence SEQ ID NO: 3. In particular, this protein is more particularly aflibercept (i.e. a protein having 100% sequence identity with the sequence SEQ ID NO: 3).

In particular, (vi) a protein having at least 85% sequence identity with the sequence SEQ ID NO: 26 comprises a protein having at least 86%, at least 87%, at least 88%, at least 89%, at least 90%, at least 91%, at least 92%, at least 93%, at least 93%, at least 94%, at least 95%, at least 96%, at least 97%, at least 98%, at least 99% and 100% sequence identity with the sequence SEQ ID NO: 26. In particular, this protein is more particularly complement factor H (i.e. a protein having 100% sequence identity with the sequence SEQ ID NO: 26).

In particular, the coding sequences for the first and second therapeutic proteins, different than one another, may for example be selected from the group consisting of:
(i) a nucleotide sequence coding for transferrin, in particular a nucleotide sequence having at least 75% sequence identity with the sequence SEQ ID NO: 15, and is in particular the sequence SEQ ID NO: 16;
(ii) a nucleotide sequence coding for a protein having antifibrotic properties, such as the protein BMP7 (Bone Morphogenic Protein 7), a protein of the anti TGF-beta type, a protein of the anti FGF2 type, a protein of the anti CTGF type (CTGF: connective tissue growth factor), and in particular a protein coding for decorin, more particularly a nucleotide sequence having at least 70% sequence identity with the sequence SEQ ID NO: 6, more particularly a sequence selected from the group consisting of the sequences SEQ ID NO: 7, SEQ ID NO: 9, SEQ ID NO: 10, SEQ ID NO: 11 and SEQ ID NO: 12, and is in particular the sequence SEQ ID NO: 7 or the sequence SEQ ID NO: 11, and more particularly the sequence SEQ ID NO: 11;
(iii) a nucleotide sequence coding for a protein having anti-inflammatory properties, in particular a protein of the anti-TNF type, such as for example hTNFR-Is, hTNFR-Is/mlgG1, Lenercept or the fusion protein comprising the extracellular domain of the human receptor p55 to TNF alpha coupled via a hinge to the constant fragment of human immunoglobulin IgG1, more particularly a sequence coding for a protein of the anti TNF-alpha type, in particular a fusion protein comprising the extracellular domain of the human receptor p55 to TNF alpha coupled via a hinge to the constant fragment of human immunoglobulin IgG1, and more particularly a sequence having at least 85% sequence identity with the sequence SEQ ID NO: 21;
(iv) a nucleotide sequence coding for a protein of the anti-VEGF type, such as S-Flt1, aflibercept, conbercept, brolucizumab, and in particular a sequence coding for aflibercept, more particularly a sequence having at least 75% sequence identity with the sequence SEQ ID NO: 1, in particular the sequence SEQ ID NO: 2;
(v) a sequence coding for a protein having antiangiogenic properties, such as angiostatin, endostatin, thrombospondin, a protein of the anti angiopoietin-2 type, a protein of the anti FGF2 type, a protein of the anti PLGF type, a protein of the anti PDGF type; and
(vi) a sequence coding for a protein regulating the activation of complement, such as complement factor I (CFI), and complement factor H, and in particular a sequence coding for complement factor HI, more particularly a sequence having at least 75% sequence identity with the sequence SEQ ID NO: 25.

“Sequences coding for the first and the second therapeutic proteins” is not to be understood as “first nucleotide sequence” and “second nucleotide sequence” as indicated previously, but rather the sequences that are present within the first nucleotide sequence and within the second nucleotide sequence according to the invention, and which code specifically for the first and the second therapeutic proteins.

According to one embodiment, the first or the second therapeutic protein of a DNA construct according to the invention is a protein having at least 85% sequence identity with the sequence SEQ ID NO: 17, this protein more particularly being transferrin.

In particular, a DNA construct according to the invention may be characterized in that the first nucleotide sequence or the second nucleotide sequence codes for:
a protein having at least 85% sequence identity with the sequence SEQ ID NO: 17, this protein more particularly being transferrin; and
a signal peptide of peptide sequence SEQ ID NO: 18.

Thus, one of the sequences coding for the first and the second therapeutic proteins of a DNA construct according to the invention may in particular be a nucleotide sequence coding for transferrin, and in particular a sequence having at least 75% sequence identity with the sequence SEQ ID NO: 15, and is in particular the sequence SEQ ID NO: 16.

In particular, a DNA construct according to the invention may be characterized in that the first nucleotide sequence or the second nucleotide sequence comprises:
a nucleotide sequence coding for transferrin, and in particular a sequence having at least 75% sequence identity with the sequence SEQ ID NO: 15, and is in particular the sequence SEQ ID NO: 16; and
the nucleotide sequence SEQ ID NO: 20 coding for a signal peptide.

According to one embodiment, the first and the second therapeutic proteins encoded by a DNA construct according to the invention are respectively:
a protein having at least 85% sequence identity with the sequence SEQ ID NO: 17, this protein more particularly being transferrin; and
a protein of the anti-TNF-alpha type, in particular a protein having at least 85% sequence identity with the sequence SEQ ID NO: 22, and more particularly the fusion protein comprising the extracellular domain of the human receptor p55 to TNF alpha coupled via a hinge to the constant fragment of human immunoglobulin IgG1 of sequence SEQ ID NO: 22.

Thus, according to one embodiment, the sequences coding for the first and the second therapeutic proteins of a DNA construct according to the invention are respectively:
a nucleotide sequence coding for transferrin, and in particular a sequence having at least 75% sequence identity with the sequence SEQ ID NO: 15, and is in particular the sequence SEQ ID NO: 16; and
a sequence coding for a protein of the anti-INF-alpha type, in particular a sequence coding for the fusion protein comprising the extracellular domain of the human receptor p55 to TNF alpha coupled via a hinge to the constant fragment of human immunoglobulin IgG1, of peptide sequence SEQ ID NO: 22, and in particular a nucleotide sequence having at least 85% sequence identity with the sequence SEQ ID NO: 21.

Inflammation and oxidative stress are important components of retinal degeneration such as ARMD) or glaucomatous neuropathy following an increase in intraocular pressure caused by glaucoma. In particular, an increase in the intraocular concentrations of TNF alpha is observed in glaucomatous eyes (Tezel et al., 2001, Invest Ophthalmol Vis Sci. 2001 July; 42(8): 1787-94) and injection of TNF-alpha in the eye of a rodent induces an axonal degeneration of the optic nerve and programmed death of the ganglion cells of the retina (Kitaoka 2006, Invest Ophthalmol Vis Sci. 2006; 47: 1448-1457). An increase in expression of the genes regulating the level of iron has also been observed in glaucomatous eyes suggesting that the oxidative stress induced by iron may play a role in the pathogenesis of glaucoma (Farkas et al., 2004). The administration of an anti-TNF and an iron chelating agent, such as transferrin, makes it possible advantageously to reduce both the inflammation and the oxidative stress mediated by iron.

In particular, a DNA construct according to the invention may be characterized in that:
(c) the first nucleotide sequence comprises:
   a nucleotide sequence coding for transferrin, and in particular a sequence having at least 75% sequence identity with the sequence SEQ ID NO: 15, and is in particular the sequence SEQ ID NO: 16; and
   the nucleotide sequence SEQ ID NO: 20 coding for a signal peptide; and
(f) the second nucleotide sequence comprises:
   a nucleotide sequence coding for a protein of the anti-TNF-alpha type, in particular a sequence coding for the fusion protein comprising the extracellular domain of the human receptor p55 to TNF alpha coupled via a hinge to the constant fragment of human immunoglobulin IgG1, of peptide sequence SEQ ID NO: 22, and in particular a nucleotide sequence having at least 85% sequence identity with the sequence SEQ ID NO: 21, and in particular the sequence SEQ ID NO: 21; and
   the nucleotide sequence SEQ ID NO: 23 coding for a signal peptide.

In particular, a DNA construct according to the invention may be characterized in that:
(c) the first nucleotide sequence codes for:
   a protein having at least 85% sequence identity with the sequence SEQ ID NO: 17, this protein more particularly being transferrin; and
   a signal peptide of peptide sequence SEQ ID NO: 18; and
(f) the second nucleotide sequence codes for:
   a protein having at least 85% sequence identity with the sequence SEQ ID NO: 22, this protein more particularly being the fusion protein comprising the extracellular domain of the human receptor p55 to TNF alpha coupled via a hinge to the constant fragment of human immunoglobulin IgG1 of sequence SEQ ID NO: 22; and
   a signal peptide of peptide sequence SEQ ID NO: 23.

According to another embodiment, the first and the second therapeutic proteins encoded by a DNA construct according to the invention are respectively:
a protein having at least 85% sequence identity with the sequence SEQ ID NO: 17, this protein more particularly being transferrin; and
a protein having antifibrotic properties, in particular a protein having at least 85% sequence identity with the sequence SEQ ID NO: 8, this protein more particularly being decorin.

Thus, according to one embodiment, the sequences coding for the first and the second therapeutic proteins of a DNA construct according to the invention are respectively:
a nucleotide sequence coding for transferrin, and in particular a sequence having at least 75% sequence identity with the sequence SEQ ID NO: 15, and is in particular the sequence SEQ ID NO: 16; and
a nucleotide sequence coding for a protein having antifibrotic properties, such as the protein BMP7 (Bone Morphogenic Protein 7), a protein of the anti TGF-beta type, a protein of the anti FGF2 type, a protein of the anti CTGF type (connective tissue growth factor), and in particular a protein coding for decorin, more particularly a nucleotide sequence having at least 70% sequence identity with the sequence SEQ ID NO: 6, more particularly a sequence selected from the group consisting of the sequences SEQ ID NO: 7, SEQ ID NO: 9, SEQ ID NO: 10, SEQ ID NO: 11 and SEQ ID NO: 12, and is in particular the sequence SEQ ID NO: 7 or the sequence SEQ ID NO: 11, and more particularly the sequence SEQ ID NO: 11.

Glaucoma, in particular primary open-angle glaucoma, is characterized by an increase in intraocular pressure following fibrosis of the trabecular network, and by loss of ganglion cells of the retina and degeneration of the optic nerve. The current treatments for glaucoma reduce the intraocular pressure but do not allow the evolution of neurodegeneration to be halted. The administration of an agent with neuroprotective action, such as an anti-TNF or transferrin, advantageously makes it possible to potentiate the effects of an antifibrotic such as decorin, and both reduce the intraocular pressure and protect the retina and the optic nerve from degeneration.

In particular, a DNA construct according to the invention may be characterized in that:
(c) the first nucleotide sequence comprises:
   a nucleotide sequence coding for transferrin, and in particular a sequence having at least 75% sequence identity with the sequence SEQ ID NO: 15, and is in particular the sequence SEQ ID NO: 16; and
   the nucleotide sequence SEQ ID NO: 20 coding for a signal peptide; and
(f) the second nucleotide sequence comprises:
   a nucleotide sequence coding for decorin, more particularly a nucleotide sequence having at least 70% sequence identity with the sequence SEQ ID NO: 6, more particularly a sequence selected from the group consisting of the sequences SEQ ID NO: 7, SEQ ID NO: 9, SEQ ID NO: 10, SEQ ID NO: 11 and SEQ ID NO: 12, and is in particular the sequence SEQ ID NO: 7 or the sequence SEQ ID NO: 11, and more particularly the sequence SEQ ID NO: 11; and
   the nucleotide sequence SEQ ID NO: 14 coding for a signal peptide.

In particular, a DNA construct according to the invention may be characterized in that:
(c) the first nucleotide sequence codes for:
   a protein having at least 85% sequence identity with the sequence
SEQ ID NO: 17, this protein more particularly being transferrin; and
   a signal peptide of peptide sequence SEQ ID NO: 18; and
(f) the second nucleotide sequence codes for:
   a protein having at least 85% sequence identity with the sequence SEQ ID NO: 8, this protein more particularly being decorin; and
   a signal peptide of peptide sequence SEQ ID NO: 13.

According to one embodiment, the first or the second therapeutic protein of a DNA construct according to the invention is a protein of the anti-VEGF type, in particular a protein having at least 85% sequence identity with the sequence SEQ ID NO: 3, this protein more particularly being aflibercept.

In particular, a DNA construct according to the invention may be characterized in that the first nucleotide sequence or the second nucleotide sequence codes for:
a protein having at least 85% sequence identity with the sequence SEQ ID NO: 3, this protein more particularly being aflibercept; and
a signal peptide of peptide sequence SEQ ID NO: 4.

Thus, one of the sequences coding for the first and the second therapeutic proteins of a DNA construct according to the invention may in particular be a nucleotide sequence coding for a protein of the anti-VEGF type, such as S-Flt1, aflibercept, conbercept, brolucizumab, and in particular a sequence coding for aflibercept, more particularly a sequence having at least 75% sequence identity with the sequence SEQ ID NO: 1, and is in particular the sequence SEQ ID NO: 2.

In particular, a DNA construct according to the invention may be characterized in that the first nucleotide sequence or the second nucleotide sequence comprises:
a nucleotide sequence coding for aflibercept, and in particular a sequence having at least 75% sequence identity with the sequence SEQ ID NO: 1, and is in particular the sequence SEQ ID NO: 2; and
the nucleotide sequence SEQ ID NO: 5 coding for a signal peptide.

According to another embodiment, the first and the second therapeutic proteins of a DNA construct according to the invention are respectively:
a protein having antifibrotic properties, in particular a protein having at least 85% sequence identity with the sequence SEQ ID NO: 8, this protein more particularly being decorin; and
a protein of the anti-VEGF type, in particular a protein having at least 85% sequence identity with the sequence SEQ ID NO: 3, this protein more particularly being aflibercept.

Thus, according to one embodiment, the sequences coding for the first and the second therapeutic proteins of a DNA construct according to the invention are respectively:
a nucleotide sequence coding for a protein having antifibrotic properties, such as the protein BMP7 (Bone Morphogenic Protein 7), a protein of the anti TGF-beta type, a protein of the anti FGF2 type, a protein of the anti CTGF type (connective tissue growth factor), and in particular a protein coding for decorin, more particularly a nucleotide sequence having at least 70% sequence identity with the sequence SEQ ID NO: 6, more particularly a sequence selected from the group consisting of the sequences SEQ ID NO: 7, SEQ ID NO: 9, SEQ ID NO: 10, SEQ ID NO: 11 and SEQ ID NO: 12, and is in particular the sequence SEQ ID NO: 7 or the sequence SEQ ID NO: 11; and
a nucleotide sequence coding for a protein of the anti-VEGF type, such as S-Flt1, aflibercept, conbercept, brolucizumab, and in particular a sequence coding for aflibercept, more particularly a sequence having at least 75% sequence identity with the sequence SEQ ID NO: 1, in particular the sequence SEQ ID NO: 2.

The presence of the antifibrotic active ingredient advantageously makes it possible to potentiate the effects of the anti-VEGF, thus improving the efficacy of this compound in the treatment of the target ocular pathologies. In particular, it was observed that even in patients receiving injections of anti-VEGF at optimal intervals, development of a subretinal fibrosis appears with time in more than half of the patients, reducing the efficacy of the anti-VEGFs with the passage of time (Daniel et al. 2014, Ophthalmology 121, 656-666). Moreover, development of subretinal fibrosis was identified as a cause of poor therapeutic response to anti-VEGFs in ARMD patients not responding to anti-VEGFs (Cohen et al. 2012, Retina 32, 1480-1485).

In particular, a DNA construct according to the invention may be characterized in that:
(c) the first nucleotide sequence comprises:
   a nucleotide sequence coding for aflibercept, and in particular a sequence having at least 75% sequence identity with the sequence SEQ ID NO: 1, and is in particular the sequence SEQ ID NO: 2; and
   the nucleotide sequence SEQ ID NO: 5 coding for a signal peptide; and
(f) the second nucleotide sequence comprises:
   a nucleotide sequence coding for decorin, more particularly a nucleotide sequence having at least 70% sequence identity with the sequence SEQ ID NO: 6, more particularly a sequence selected from the group consisting of the sequences SEQ ID NO: 7, SEQ ID NO: 9, SEQ ID NO: 10, SEQ ID NO: 11 and SEQ ID NO: 12, and is in particular the sequence SEQ ID NO: 7 or the sequence SEQ ID NO: 11; and
   the nucleotide sequence SEQ ID NO: 14 coding for a signal peptide.

In particular, a DNA construct according to the invention may be characterized in that:
(c) the first nucleotide sequence comprises:
   a nucleotide sequence coding for aflibercept, and in particular a sequence having at least 75% sequence identity with the sequence SEQ ID NO: 1, and is in particular the sequence SEQ ID NO: 2; and
   the nucleotide sequence SEQ ID NO: 5 coding for a signal peptide; and
(f) the second nucleotide sequence comprises:
   a nucleotide sequence coding for decorin, more particularly the sequence SEQ ID NO: 7; and
   the nucleotide sequence SEQ ID NO: 14 coding for a signal peptide.

In particular, a DNA construct according to the invention may be characterized in that:
(c) the first nucleotide sequence comprises:
   a nucleotide sequence coding for aflibercept, and in particular a sequence having at least 75% sequence identity with the sequence SEQ ID NO: 1, and is in particular the sequence SEQ ID NO: 2; and
   the nucleotide sequence SEQ ID NO: 5 coding for a signal peptide; and
(f) the second nucleotide sequence comprises:
   a nucleotide sequence coding for decorin, more particularly the sequence SEQ ID NO: 11; and
   the nucleotide sequence SEQ ID NO: 14 coding for a signal peptide.

In particular, a DNA construct according to the invention may be characterized in that:
(c) the first nucleotide sequence codes for:
   a protein having at least 85% sequence identity with the sequence SEQ ID NO: 3, this protein more particularly being aflibercept; and
   a signal peptide of peptide sequence SEQ ID NO: 4; and
(f) the second nucleotide sequence codes for:
   a protein having at least 85% sequence identity with the sequence SEQ ID NO: 8, this protein more particularly being decorin; and
   a signal peptide of peptide sequence SEQ ID NO: 13.

According to another embodiment, the first and the second therapeutic proteins of a DNA construct according to the invention are respectively:
a protein of the anti-TNF-alpha type, in particular a protein having at least 85% sequence identity with the sequence SEQ ID NO: 22, and more particularly the fusion protein comprising the extracellular domain of the human receptor p55 to TNF alpha coupled via a hinge to the constant fragment of human immunoglobulin IgG1 of sequence SEQ ID NO: 22;
a protein of the anti-VEGF type, in particular a protein having at least 85% sequence identity with the sequence SEQ ID NO: 3, this protein more particularly being aflibercept.

Thus, according to one embodiment, the sequences coding for the first and the second therapeutic proteins of a DNA construct according to the invention are respectively:
a sequence coding for a protein of the anti-TNF-alpha type, in particular a sequence coding for the fusion protein comprising the extracellular domain of the human receptor p55 to TNF alpha coupled via a hinge to the constant fragment of human immunoglobulin IgG1 of peptide sequence SEQ ID NO: 22, and in particular a nucleotide sequence having at least 85% sequence identity with the sequence SEQ ID NO: 21; and
a nucleotide sequence coding for a protein of the anti-VEGF type, such as S-Flt1, aflibercept, conbercept, brolucizumab, and in particular a sequence coding for aflibercept, more particularly a sequence having at least 75% sequence identity with the sequence SEQ ID NO: 1, and is in particular the sequence SEQ ID NO: 2.

The presence of the anti-INF-alpha active ingredient makes it possible advantageously to potentiate the effects of the anti-VEGF, thus improving the efficacy of this compound in the treatment of the target ocular pathologies. In particular, it is well known that VEGF induces retinal permeability but inflammatory agents, such as TNF-alpha, may also lead to vascular permeability, in particular in patients who do not respond to anti-VEGF treatment, such as may be observed in patients with diabetic retinopathy (Arias L. et al.; Retina 2010, 30: 1601e1608 and Sfikakis et al.; Diabetes Care 2010, 33: 1523e1528). Recent tests suggest that VEGF and TNF-alpha induce permeability by different mechanisms.

In particular, a DNA construct according to the invention may be characterized in that:
(c) the first nucleotide sequence comprises:
   a nucleotide sequence coding for a protein of the anti-TNF-alpha type, in particular a sequence coding for the fusion protein comprising the extracellular domain of the human receptor p55 to TNF alpha coupled via a hinge to the constant fragment of human immunoglobulin IgG1, of peptide sequence SEQ ID NO: 22, and in particular a nucleotide sequence having at least 85% sequence identity with the sequence SEQ ID NO: 21, and in particular the sequence SEQ ID NO: 21; and
   the nucleotide sequence SEQ ID NO: 23 coding for a signal peptide; and
(f) the second nucleotide sequence comprises:
   a nucleotide sequence coding for aflibercept, and in particular a sequence having at least 75% sequence identity with the sequence SEQ ID NO: 1, and is in particular the sequence SEQ ID NO: 2; and
   the nucleotide sequence SEQ ID NO: 5 coding for a signal peptide.

In particular, a DNA construct according to the invention may be characterized in that:
(c) the first nucleotide sequence codes for:
   a protein having at least 85% sequence identity with the sequence SEQ ID NO: 22, this protein more particularly being the fusion protein comprising the extracellular domain of the human receptor p55 to TNF alpha coupled via a hinge to the constant fragment of human immunoglobulin IgG1 of sequence SEQ ID NO: 22; and
   a signal peptide of peptide sequence SEQ ID NO: 23; and
(f) the second nucleotide sequence codes for:
   a protein having at least 85% sequence identity with the sequence SEQ ID NO: 3, this protein more particularly being aflibercept; and
   a signal peptide of peptide sequence SEQ ID NO: 4.

According to another embodiment, the first and the second therapeutic proteins of a DNA construct according to the invention are respectively:
a protein regulating the activation of complement, in particular a protein having at least 85% sequence identity with the sequence SEQ ID NO: 26, this protein more particularly being complement factor H;
a protein of the anti-VEGF type, in particular a protein having at least 85% sequence identity with the sequence SEQ ID NO: 3, this protein more particularly being aflibercept.

Thus, according to one embodiment, the sequences coding for the first and the second therapeutic proteins of a DNA construct according to the invention are respectively:
a nucleotide sequence coding for a protein regulating the activation of complement, such as complement factor I (CFI), and complement factor H, and in particular a sequence coding for complement factor H, more particularly a sequence having at least 75% sequence identity with the sequence SEQ ID NO: 25; and
a nucleotide sequence coding for a protein of the anti-VEGF type, such as S-Flt1, aflibercept, conbercept, brolucizumab, and in particular a sequence coding for aflibercept, more particularly a sequence having at least 75% sequence identity with the sequence SEQ ID NO: 1, and is in particular the sequence SEQ ID NO: 2.

Activation of the complement alternative pathway is an important component of ARMD. This activation leads to formation of the membrane attack complex, recruitment of macrophages, and induction of inflammation with production of cytokines involved in the inflammasome. Complement factor H is involved in regulating the autoactivation of complement. Several polymorphic variants in the gene coding for CFH, affecting the function of the protein, confer strong susceptibility for developing the two forms of ARMD, wet and dry. Conversely, inhibition of the alternative pathway by intraocular injection of CFH reduces neovascularization in animal models of choroid neovascularization. Thus, administration of an active ingredient regulating the activation of complement and of an anti-VEGF, such as aflibercept, advantageously makes it possible to reduce both the neovascularization and the inflammation associated with ARMD.

In particular, a DNA construct according to the invention may be characterized in that:
(c) the first nucleotide sequence comprises:
   a nucleotide sequence coding for a protein regulating the activation of complement, such as complement factor I (CFI), and complement factor H, and in particular a sequence coding for complement factor H, more particularly a sequence having at least 75% sequence identity with the sequence SEQ ID NO: 25; and
   the nucleotide sequence SEQ ID NO: 28 coding for a signal peptide; and
(f) the second nucleotide sequence comprises:
   a nucleotide sequence coding for aflibercept, and in particular a sequence having at least 75% sequence identity with the sequence SEQ ID NO: 1, and is in particular the sequence SEQ ID NO: 2; and
   the nucleotide sequence SEQ ID NO: 5 coding for a signal peptide.

In particular, a DNA construct according to the invention may be characterized in that:
(c) the first nucleotide sequence codes for:
   a protein regulating the activation of complement, in particular a protein having at least 85% sequence identity with the sequence SEQ ID NO: 26, this protein more particularly being complement factor H; and
   a signal peptide of peptide sequence SEQ ID NO: 27; and
(f) the second nucleotide sequence codes for:
   a protein having at least 85% sequence identity with the sequence SEQ ID NO: 3, this protein more particularly being aflibercept; and
   a signal peptide of peptide sequence SEQ ID NO: 4.

Moreover, a DNA construct according to the invention is characterized in that the first nucleotide sequence also codes for a signal peptide allowing secretion of the first therapeutic protein.

A signal peptide of this kind is familiar to a person skilled in the art. This signal peptide may for example be human tissue plasminogen activator (tPA) of peptide sequence SEQ ID NO: 4 (and which may for example be encoded by the nucleotide sequence SEQ ID NO: 5) or the signal peptide of HTLV-1 Env of peptide sequence SEQ ID NO: 29 (and which may for example be encoded by the nucleotide sequence SEQ ID NO: 30). It may also be the native peptide signal of the therapeutic protein in question, such as for example:
the native peptide signal of decorin, of peptide sequence SEQ ID NO: 13 (and which may for example be encoded by the nucleotide sequence SEQ ID NO: 14);
the native peptide signal of transferrin, of peptide sequence SEQ ID NO: 18 (and which may for example be encoded by the nucleotide sequence SEQ ID NO: 19 or by the nucleotide sequence SEQ ID NO: 20);
the signal peptide of a protein of the anti-TNF-alpha type, in particular the native peptide signal fusion protein comprising the extracellular domain of the human receptor p55 to TNF alpha coupled via a hinge to the constant fragment of human immunoglobulin IgG1, this signal peptide having a peptide sequence SEQ ID NO: 23 (and which may for example be encoded by the nucleotide sequence SEQ ID NO: 24); or
the native peptide signal of factor H, of peptide sequence SEQ ID NO: 27 (and which may for example be encoded by the nucleotide sequence SEQ ID NO: 28).

As mentioned above, this signal peptide is contiguous with the first therapeutic protein, i.e. it is fused directly at the N-terminal of the first therapeutic protein, and therefore the sequence coding for the signal peptide is at 5′ of the sequence coding for the first therapeutic protein.

Moreover, a DNA construct according to the invention is characterized in that the second nucleotide sequence also codes for a signal peptide allowing secretion of the second therapeutic protein.

This signal peptide may for example be human tissue plasminogen activator (tPA) of peptide sequence SEQ ID NO: 4 (and which may for example be encoded by the nucleotide sequence SEQ ID NO: 5) or the signal peptide of HTLV-1 Env of peptide sequence SEQ ID NO: 29 (and which may for example be encoded by the nucleotide sequence SEQ ID NO: 30). It may also be the native peptide signal of the therapeutic protein in question, such as for example:
the native peptide signal of decorin, of peptide sequence SEQ ID NO: 13 (and which may for example be encoded by the nucleotide sequence SEQ ID NO: 14);
the native peptide signal of transferrin, of peptide sequence SEQ ID NO: 18 (and which may for example be encoded by the nucleotide sequence SEQ ID NO: 19 or by the nucleotide sequence SEQ ID NO: 20);
the signal peptide of a protein of the anti-TNF-alpha type, in particular the native peptide signal fusion protein comprising the extracellular domain of the human receptor p55 to TNF alpha coupled via a hinge to the constant fragment of human immunoglobulin IgG1, this signal peptide having a peptide sequence SEQ ID NO: 23 (and which may for example be encoded by the nucleotide sequence SEQ ID NO: 24); or
the native peptide signal of factor HI, of peptide sequence SEQ ID NO: 27 (and which may for example be encoded by the nucleotide sequence SEQ ID NO: 28).

This signal peptide may be identical to or different than the signal peptide encoded by the first nucleotide sequence, and is preferably different than the signal peptide encoded by the first nucleotide sequence.

As mentioned above, this signal peptide is contiguous with the second therapeutic protein, i.e. it is fused directly at the N-terminal of the second therapeutic protein, and therefore the sequence coding for the signal peptide is at 5′ of the sequence coding for the second therapeutic protein.

According to a particular embodiment, the signal peptide encoded by the first nucleotide sequence and the signal peptide encoded by the second nucleotide sequence are, independently of one another, selected from the group consisting of the peptide sequences SEQ ID NO: 4; SEQ ID NO: 13, SEQ ID NO: 18, SEQ ID NO: 23, SEQ ID NO: 27 and SEQ ID NO: 29.

According to one embodiment, the sequence coding for the signal peptide encoded by the first nucleotide sequence and the sequence coding for the signal peptide encoded by the second nucleotide sequence are, independently of one another, selected from the group consisting of the nucleotide sequences SEQ ID NO: 5; SEQ ID NO: 14; SEQ ID NO: 19, SEQ ID NO: 20; SEQ ID NO: 24, SEQ ID NO: 28 and SEQ ID NO: 30.

Moreover, a DNA construct according to the invention is also characterized in that it comprises (d) a promoter allowing expression of the first therapeutic protein of a construct according to the invention as well as (g) a promoter allowing expression of the second therapeutic protein of a construct according to the invention.

These promoters may be identical or different. According to a particular embodiment, these two promoters are different than one another.

Said promoters may for example be promoters of the CAG or CMV type.

Moreover, a DNA construct according to the invention is characterized in that it comprises (e) a polyadenylation sequence at 3′ of the first nucleotide sequence and (h) a polyadenylation sequence at 3′ of the second nucleotide sequence.

A polyadenylation sequence contains in particular a conserved motif of sequence AATAAA, familiar to a person skilled in the art.

These two polyadenylation sequences may be identical to or different than one another.

According to one embodiment, they are different than one another.

These polyadenylation sequences may for example be polyadenylation sequences of the type RBG (Rabbit Beta Globin), or BGH (Bovine Growth Hormone).

Finally, as mentioned above, a DNA construct according to the invention is administered to the patient by injection into a ciliary muscle and then electrotransfer into the cells of the ciliary muscle.

In a particular embodiment, the DNA construct according to the invention is of circular shape.

In one embodiment of the invention, the DNA construct is a naked DNA construct.

According to one embodiment of the invention, the DNA construct according to the invention is a naked DNA construct of circular shape.

In one embodiment of the invention, the DNA construct according to the invention is a naked DNA construct of circular shape in which the sequences coding for the first and the second therapeutic proteins of a DNA construct according to the invention are respectively:
a nucleotide sequence coding for transferrin, and in particular a sequence having at least 75% sequence identity with the sequence SEQ ID NO: 15, and is in particular the sequence SEQ ID NO: 16; and
a sequence coding for a protein of the anti-INF-alpha type, in particular a sequence coding for the fusion protein comprising the extracellular domain of the human receptor p55 to TNF alpha coupled via a hinge to the constant fragment of human immunoglobulin IgG1 of peptide sequence SEQ ID NO: 22, and in particular a nucleotide sequence having at least 85% sequence identity with the sequence SEQ ID NO: 21.

In one embodiment of the invention, the DNA construct according to the invention is a naked DNA construct of circular shape in which the sequences coding for the first and the second therapeutic proteins of a DNA construct according to the invention are respectively:
a nucleotide sequence coding for a protein having antifibrotic properties, such as the protein BMP7 (Bone Morphogenic Protein 7), a protein of the anti TGF-beta type, a protein of the anti FGF2 type, a protein of the anti CTGF type (connective tissue growth factor), and in particular a protein coding for decorin, more particularly a nucleotide sequence having at least 70% sequence identity with the sequence SEQ ID NO: 6, more particularly a sequence selected from the group consisting of the sequences SEQ ID NO: 7, SEQ ID NO: 9, SEQ ID NO: 10, SEQ ID NO: 11 and SEQ ID NO: 12, and is in particular the sequence SEQ ID NO: 7 or the sequence SEQ ID NO: 11, and more particularly the sequence SEQ ID NO: 11; and
a nucleotide sequence coding for a protein of the anti-VEGF type, such as S-Flt1, aflibercept, conbercept, brolucizumab, and in particular a sequence coding for aflibercept, more particularly a sequence having at least 75% sequence identity with the sequence SEQ ID NO: 1, and is in particular the sequence SEQ ID NO: 2.

In one embodiment, the DNA construct according to the invention is a naked DNA construct of circular shape in which the sequences coding for the first and the second therapeutic proteins of a DNA construct according to the invention are respectively:
a nucleotide sequence coding for transferrin, and in particular a sequence having at least 75% sequence identity with the sequence SEQ ID NO: 15, and is in particular the sequence SEQ ID NO: 16; and
a nucleotide sequence coding for a protein having antifibrotic properties, such as the protein BMP7 (Bone Morphogenic Protein 7), a protein of the anti TGF-beta type, a protein of the anti FGF2 type, a protein of the anti CTGF type (connective tissue growth factor), and in particular a protein coding for decorin, more particularly a nucleotide sequence having at least 70% sequence identity with the sequence SEQ ID NO: 6, more particularly a sequence selected from the group consisting of the sequences SEQ ID NO: 7, SEQ ID NO: 9, SEQ ID NO: 10, SEQ ID NO: 11 and SEQ ID NO: 12, and is in particular the sequence SEQ ID NO: 7 or the sequence SEQ ID NO: 11, and more particularly the sequence SEQ ID NO: 11.

In one embodiment according to the invention, the DNA construct according to the invention is a naked DNA construct of circular shape in which the sequences coding for the first and the second therapeutic proteins of a DNA construct according to the invention are respectively:
a sequence coding for a protein of the anti-INF-alpha type, in particular a sequence coding for the fusion protein comprising the extracellular domain of the human receptor p55 to TNF alpha coupled via a hinge to the constant fragment of human immunoglobulin IgG1 of protein sequence SEQ ID NO: 22, and in particular a nucleotide sequence having at least 85% sequence identity with the sequence SEQ ID NO: 21; and
a nucleotide sequence coding for a protein of the anti-VEGF type, such as S-Flt1, aflibercept, conbercept, brolucizumab, and in particular a sequence coding for aflibercept, more particularly a sequence having at least 75% sequence identity with the sequence SEQ ID NO: 1, and is in particular the sequence SEQ ID NO: 2.

In one embodiment according to the invention, the DNA construct according to the invention is a naked DNA construct of circular shape in which the sequences coding for the first and the second therapeutic proteins of a DNA construct according to the invention are respectively:
a nucleotide sequence coding for a protein regulating the activation of complement, such as complement factor I (CFI), and complement factor H, and in particular a sequence coding for complement factor H, more particularly a sequence having at least 75% sequence identity with the sequence SEQ ID NO: 25; and
a nucleotide sequence coding for a protein of the anti-VEGF type, such as S-Flt1, aflibercept, conbercept, brolucizumab, and in particular a sequence coding for aflibercept, more particularly a sequence having at least 75% sequence identity with the sequence SEQ ID NO: 1, and is in particular the sequence SEQ ID NO: 2.

The present invention also relates to a DNA construct intended for the nonviral transfer of nucleic acids into the muscle cells of a patient's ocular sphere for treating ocular pathologies, characterized in that it comprises:
(a) a bacterial or prokaryotic origin of replication, in particular bacterial,
(b) one or more sequences promoting the expression of DNA in the patient's ocular sphere,
(c) a first nucleotide sequence coding for:
   a first therapeutic protein, said first therapeutic protein being aflibercept; and
   a signal peptide allowing secretion of this first therapeutic protein,
this signal peptide being contiguous with the sequence of the first therapeutic protein, at the N-terminal of said first therapeutic protein,
(d) a promoter allowing expression of this first therapeutic protein in the patient's ocular sphere;
(e) a polyadenylation sequence at 3′ of the first nucleotide sequence;
(f) a second nucleotide sequence coding for:
   a second therapeutic protein, said second therapeutic protein being decorin, and
   a signal peptide allowing secretion of this second therapeutic protein, the signal peptide being contiguous with the sequence of the second therapeutic protein, at the N-terminal of said second therapeutic protein; and
(g) a promoter allowing expression of this second therapeutic protein in the patient's ocular sphere; and
(h) a polyadenylation sequence at 3′ of the second nucleotide sequence.

### Application of a DNA Construct Described Above

A DNA construct as defined above is in particular intended for the treatment of ocular pathologies.

An ocular pathology according to the present invention is a retinal degeneration.

This retinal degeneration may in particular be selected from the group consisting of wet or dry age-related macular degeneration (ARMD); diabetic retinopathies (DR); a retinal venous occlusion, in particular a central retinal vein occlusion (CRVO) or a branch retinal vein occlusion (BRVO); a myopic choroid neovascularization (CNV); a uveitis, in particular a noninfectious uveitis; a retinitis pigmentosa and a glaucoma.

Diabetic retinopathy is intended in particular to denote a decline of visual acuity due to diabetic macular edema (DME), an intravitreal hemorrhage, a retinal detachment, or a neovascular glaucoma.

According to a particular embodiment, and especially when a construct according to the invention used for treating an ocular pathology comprises, as first and second therapeutic proteins, aflibercept and decorin as defined above, said ocular pathology is a retinal degeneration that may more particularly be selected from the group consisting of age-related macular degeneration (ARMD), in particular the wet form; diabetic retinopathies (DR); a retinal venous occlusion, in particular a central retinal vein occlusion (CRVO) or a branch retinal vein occlusion (BRVO); and a myopic choroid neovascularization (CNV);

and more particularly may be selected from the group consisting of age-related macular degeneration (ARMD), in particular the (wet) neovascular form of ARMD; a decline of visual acuity due to diabetic macular edema (DME); a retinal venous occlusion, in particular a central retinal vein occlusion (CRVO) or a branch retinal vein occlusion (BRVO); and a myopic choroid neovascularization (CNV).

Diabetic retinopathy is intended in particular to denote a decline of visual acuity due to diabetic macular edema (DME) and the formation of neovasculature observed in the proliferative form of diabetic retinopathy.

As stated above, a DNA construct according to the invention is injected into an ocular muscle, the ciliary muscle, where it is submitted to electrotransfer. The known technique of nonviral gene therapy used in the present invention is injection of the DNA construct into an ocular muscle and then electrotransfer to induce a transient permeabilization of the cells of the ciliary muscle and migration of the DNA to optimize transfection of the DNA construct. This technique of electrotransfer of DNA (also called electroporation or electropermeabilization) is easy to apply, reliable and safe for the patient. In contrast to viral vectors, electrotransfer of DNA does not induce an immune response and allows long-term expression of the genes thus introduced. Moreover, studies conducted on lentiviruses and retroviruses show that the latter are liable to induce insertion mutations during their integration in the host genome. The DNA constructs described here do not have drawbacks of this type, are easy to produce and manipulate and do not induce an immune response, thus making them perfectly suitable for gene therapy of patients, especially human patients.

According to the present invention, a DNA construct is injected into the ciliary muscle as the latter is capable of producing the proteins homogenously and continually and, owing to its position, promotes diffusion of these proteins in the whole ocular sphere (Blocquel et al. “Plasmid electrotransfer of eye ciliary muscle: principles and therapeutic efficacy using hTNF-alpha soluble receptor in uveitis”, FASEB J. 2006 February; 20(2): 389-91). Smooth muscle cells have a low renewal rate and are well distributed on either side of the lens. The quantity of protein to be produced is proportional to the surface area of the muscle transfected (Touchard “The ciliary smooth muscle electrotransfer: basic principles and potential for sustained intraocular production of therapeutic proteins”, J Gene Med. 2010 November; 12(11): 904-19). Thus, production of the proteins of interest according to the present invention, and in particular of therapeutic proteins as described above, will be homogeneous and constant in the whole ocular sphere and will be limited to this ocular sphere. We may mention, as an example, the method of electrotransfer described in patent application EP2266656, which relates to a method of injection of a composition that may contain DNA at the level of the tissues of the ciliary body and/or extraocular muscle tissues.

The ciliary muscle forms part of the ciliary body, near the limbus and just behind the sclera. Injection of a DNA construct according to the invention into the latter is therefore very slightly invasive in contrast to subretinal injections and therefore advantageously constitutes the injection site of the DNA construct according to the invention.

According to another aspect, the present invention also relates to the use of a DNA construct for treating an ocular pathology, said DNA construct being intended for the nonviral transfer of nucleic acids into the muscle cells of the ocular sphere of a patient with said ocular pathology; said DNA construct being characterized in that it comprises:
(a) a bacterial or prokaryotic origin of replication, in particular bacterial,
(b) one or more sequences promoting the expression of DNA in the patient's ocular sphere,
(c) a first nucleotide sequence coding for:
   a first therapeutic protein, and
   a signal peptide allowing secretion of this first therapeutic protein,
this signal peptide being contiguous with the sequence of the first therapeutic protein, at the N-terminal of said first therapeutic protein,
(d) a promoter allowing expression of this first therapeutic protein in the patient's ocular sphere;
(e) a polyadenylation sequence at 3′ of the first nucleotide sequence;
(f) a second nucleotide sequence coding for:
   a second therapeutic protein, different than the first therapeutic protein, and
   a signal peptide allowing secretion of this second therapeutic protein,
the signal peptide being contiguous with the sequence of the second therapeutic protein, at the N-terminal of said second therapeutic protein;
(g) a promoter allowing expression of this second therapeutic protein in the patient's ocular sphere, and
(h) a polyadenylation sequence at 3′ of the second nucleotide sequence;
said DNA construct being administered to the patient by injection into a ciliary muscle and then electrotransfer into the cells of the ciliary muscle.

According to another aspect, the present invention also relates to a method of treating an ocular pathology, comprising the administration of a DNA construct to a patient by injection into a ciliary muscle and then electrotransfer into the cells of the ciliary muscle, said DNA construct being intended for the nonviral transfer of nucleic acids into the muscle cells of the ocular sphere of a patient with said ocular pathology;

said DNA construct being characterized in that it comprises:
(a) a bacterial or prokaryotic origin of replication, in particular bacterial,
(b) one or more sequences promoting the expression of DNA in the patient's ocular sphere,
(c) a first nucleotide sequence coding for:
   a first therapeutic protein, and
   a signal peptide allowing secretion of this first therapeutic protein,
this signal peptide being contiguous with the sequence of the first therapeutic protein, at the N-terminal of said first therapeutic protein,
(d) a promoter allowing expression of this first therapeutic protein in the patient's ocular sphere;
(e) a polyadenylation sequence at 3′ of the first nucleotide sequence;
(f) a second nucleotide sequence coding for:
   a second therapeutic protein, different than the first therapeutic protein, and
   a signal peptide allowing secretion of this second therapeutic protein,
the signal peptide being contiguous with the sequence of the second therapeutic protein, at the N-terminal of said second therapeutic protein;
(g) a promoter allowing expression of this second therapeutic protein in the patient's ocular sphere; and
(h) a polyadenylation sequence at 3′ of the second nucleotide sequence.

The invention is described below in more detail by means of the following examples, which are presented only for purposes of illustration.

### Example 1

The inventors have demonstrated the expression of two therapeutic proteins encoded in a DNA construct according to the invention (plasmid) in the vitreous body of the eye of rats after electrotransfer of this DNA construct in the ciliary muscle.

### Plasmids

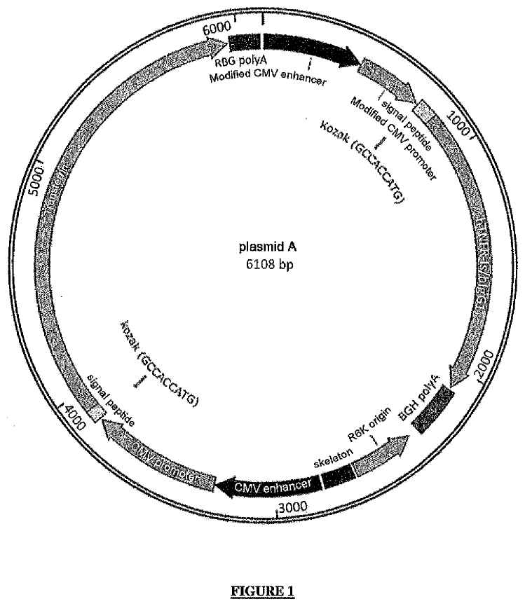

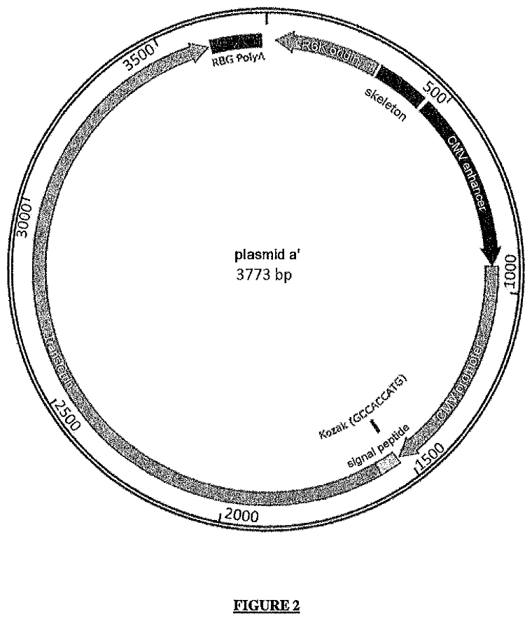

A DNA construct according to the invention, designated plasmid A, comprising:
the sequence SEQ ID NO: 16 coding for human transferrin of sequence SEQ ID NO: 17 (the sequence coding for its signal peptide being the sequence SEQ ID NO: 20);
as well as the sequence SEQ ID NO: 21 coding for a fusion protein comprising the extracellular domain of the human receptor p55 to TNF alpha coupled via a hinge to the constant fragment of human immunoglobulin IgG1 (hTNFR-Is/hIgG1) of sequence SEQ ID NO: 22 (the sequence coding for its signal peptide being the sequence SEQ ID NO: 24);
these two sequences being under the control of promoters of the CMV type;
   was first prepared by the conventional methods and is shown in FIG. 1. A comparative construct, called simple, as it only codes for a single one of the proteins of interest mentioned above, i.e. the sequence SEQ ID NO: 16 coding for human transferrin of sequence SEQ ID NO: 17, was also prepared by a similar method, with a plasmid skeleton similar to the plasmid A construct, the only sequence coding for a protein of interest also being under the control of a promoter of the CMV type. This comparative plasmid (called Plasmid a′) is shown in FIG. 2.

### Animals

Long Evans rats aged 7 weeks are used in accordance with the provisions of the ARVO protocol (Association for Research in Vision and Ophthalmology). The rats are anesthetized by intramuscular injection of a dose of ketamine (40 mg/kg) and xylazine (4 mg/kg) before bilateral injection of the plasmid (30 μg/eye) and electrotransfer on day 0 (D0). Six rats are used for each of the analysis times (D3, D7, D14, D21, and D30).

At each analysis time, the rats are euthanized by administration of a lethal dose of pentobarbital (400 mg/kg) and then the animals are enucleated and the ocular fluids (vitreous humor and aqueous humor) are taken and stored at −80° C. until analysis.

### Electrotransfer at the Level of the Ciliary Muscle of the Rat

Electrotransfer is carried out as described in Blocquel et al. “Plasmid electrotransfer of eye ciliary muscle: principles and therapeutic efficacy using hTNF-alpha soluble receptor in uveitis” (FASEB J 2006; 20:389-391), modifying the route of injection by a transscleral approach (Touchard “The ciliary smooth muscle electrotransfer: basic principles and potential for sustained intraocular production of therapeutic proteins”, J Gene Med. 2010 November; 12(11): 904-19). The plasmids are injected at a rate of 30 μg in 10 μL of Tris-EDTA NaCl solution, in the ciliary muscle of the animals using a suitable syringe.

The electrical impulses are administered by means of a special iridium/platinum electrode of 250 μm diameter. This internal electrode is introduced into the existing transscleral tunnel. The external electrode is a thin sheet of stainless steel curved to match the shape of the eye and placed at the level of the limbus opposite the internal electrode. Electrotransfer is carried out at a rate of 8 unipolar square electrical impulses (200V/cm, 10 ms, 5 Hz) generated by an electroporator similar to that described in Touchard et al. (J Gene Med. 2010 November; 12(11): 904-19).

### Test Results

Samples of ocular fluids are taken at 3 days (D+3), 7 days (D+7), 14 days (D+14), 21 days (D+21) and 30 days (D+30) after injection of plasmid followed by electrotransfer (D0). For each of these samples, an ELISA assay is carried out in order to measure the amount of human transferrin and/or of the anti-TNF-alpha fusion protein of sequence SEQ ID NO: 22 present in the samples.

A mean concentration is thus calculated for each of the groups with the passage of time.

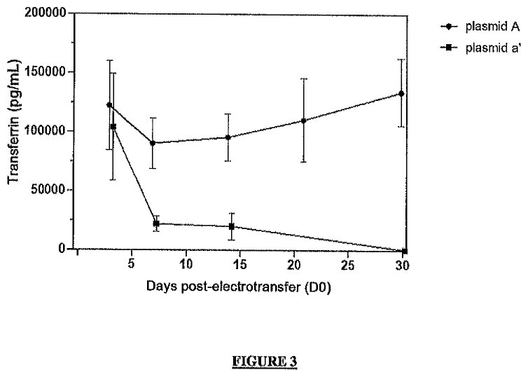

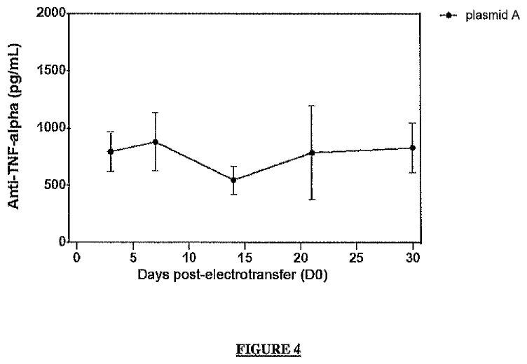

The results obtained are shown:
in FIG. 3, showing in particular the concentration of human transferrin (in pg/mL) produced in the ocular fluids taken from D+3 to D+30 by a construct according to the invention in comparison with a construct not according to the invention; and
in FIG. 4, showing the concentration of anti-TNF-alpha fusion protein (in pg/mL) of sequence SEQ ID NO: 22 produced in the ocular fluids taken from D+3 to D+30.

Examination of these figures shows that the concentration of anti-TNF-alpha fusion protein and of human transferrin is constant over time in the rats that received the construct according to the invention.

Thus, these experiments demonstrate the fact that the application of a construct according to the invention, of very large size owing to the presence of not one but two coding sequences for proteins of interest, penetrates the targeted cells effectively and allows expression of the two proteins encoded by said construct at the level of the site of interest.

Moreover, they also illustrate, quite unexpectedly, the capacity of a construct according to the invention to produce, more stably over time, the proteins that it encodes compared to constructs only coding for a single one of these proteins (FIG. 3).

### Example 2

The inventors confirmed the observations made in example 1 in a second experimental protocol using a plasmid according to the invention different than that used in example 1.

### Plasmids

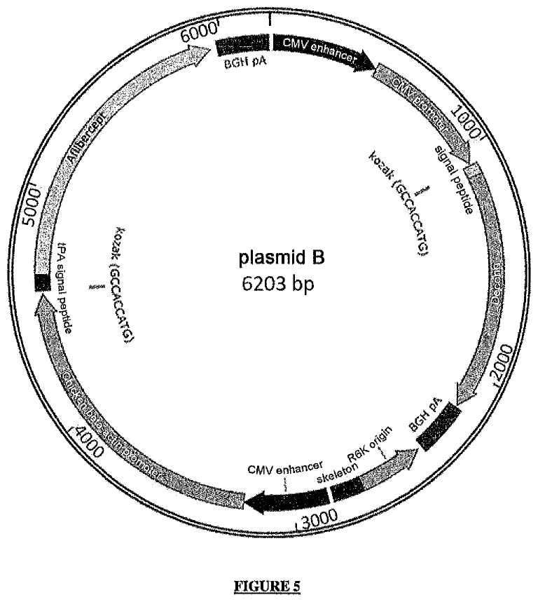

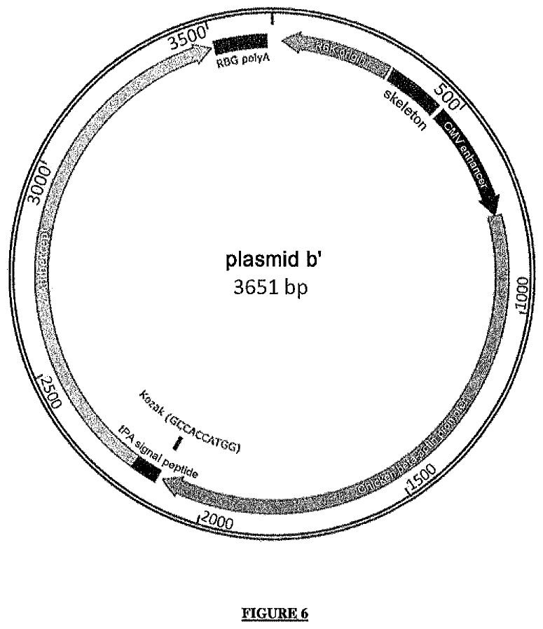

A DNA construct according to the invention, designated plasmid B, comprising:
the sequence SEQ ID NO: 7 coding for decorin of sequence SEQ ID NO: 8;
as well as the sequence SEQ ID NO: 2 coding for aflibercept of sequence SEQ ID NO: 3;
these two sequences being under the control of promoters of the CMV type and CAG type, respectively;
   was first prepared by the conventional methods and is shown in FIG. 5. A comparative construct, called simple, as it only codes for a single one of the proteins of interest mentioned above, i.e. the sequence SEQ ID NO: 2 coding for aflibercept of sequence SEQ ID NO: 3, was also prepared by a similar method, with a plasmid skeleton similar to the construct plasmid B, the single sequence coding for a protein of interest also being under the control of a promoter of the CAG type. The expression cassette of this protein of interest is thus identical in the two constructs. This comparative plasmid (called Plasmid b′) is shown in FIG. 6.

The animals used in this protocol are as described in example 1. Six rats are used for each of the analysis times (D3, D7, D14 and D21).

At each analysis time, the rats are euthanized by administration of a lethal dose of pentobarbital (400 mg/kg) and then the animals are enucleated and the ocular fluids (vitreous humor and aqueous humor) are taken and stored at −80° C. until analysis.

Electrotransfer is carried out as described in example 1.

### Test Results

Samples of ocular fluids are taken at 3 days (D+3), 7 days (D+7), 14 days (D+14) and 21 days (D+21) after injection of plasmid followed by electrotransfer (D0). For each of these samples, an ELISA assay is carried out in order to measure the amount of decorin and/or of aflibercept present in the samples.

A mean concentration is thus calculated for each of the groups with the passage of time.

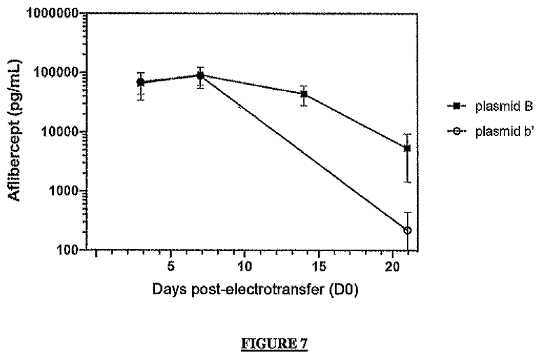

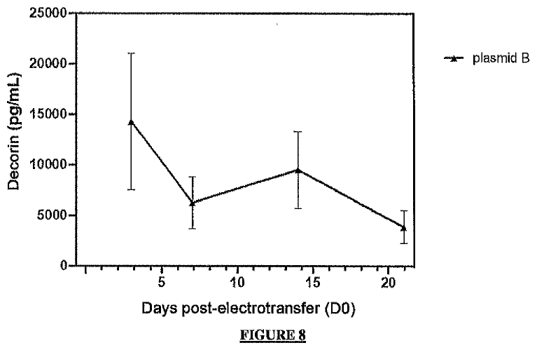

The results obtained are shown:
in FIG. 7, showing in particular the concentration of aflibercept (in pg/mL) produced in the ocular fluids taken from D+3 to D+21 by a construct according to the invention in comparison with a construct not according to the invention; and
in FIG. 8, showing the concentration of decorin (in pg/mL) produced in the ocular fluids taken from D+3 to D+21.

Examination of these figures shows that the plasmid according to the invention allows expression of the two therapeutic proteins of interest.

Thus, these experiments demonstrate the fact that the application of a construct according to the invention, of very large size owing to the presence of not one but two coding sequences for proteins of interest, penetrates the targeted cells effectively and allows expression of the two proteins encoded by said construct at the level of the site of interest.

Moreover, as had been shown above in example 1, they also illustrate, quite unexpectedly, the capacity of a construct according to the invention to produce, more stably over time, the proteins that it encodes compared to constructs only coding for a single one of these proteins (FIG. 7).

### Example 3

Moreover, the inventors also confirmed the observations made above in an experimental protocol using a plasmid according to the invention different than that used in examples 1 and 2.

### Plasmids

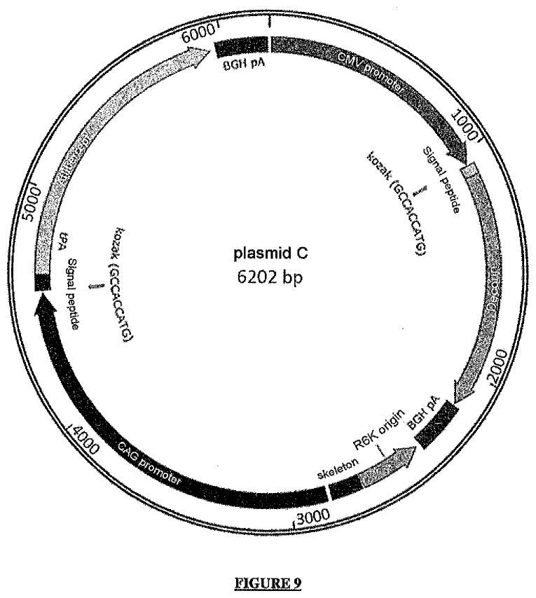

A DNA construct according to the invention, designated plasmid C, comprising:
the sequence SEQ ID NO: 11 coding for decorin of sequence SEQ ID NO: 8 under the control of a promoter of the CMV type;
as well as the sequence SEQ ID NO: 2 coding for aflibercept of sequence SEQ ID NO: 3 under the control of a promoter of the CAG type;
   was first prepared by the conventional methods and is shown in FIG. 9.

### Animals

Brown Norway rats aged from 7 to 8 weeks are used according to the provisions of the ARVO protocol (Association for Research in Vision and Ophthalmology). The rats are anesthetized by intramuscular injection of a dose of ketamine (40 mg/kg) and xylazine (4 mg/kg) before bilateral injection of the plasmid (30 μg/eye) or vehicle (10 μL) and electrotransfer on day 0 (D0). Six rats are used for each of the treatments. Electrotransfer is carried out as described in example 1.

On D3, choroid neovascularization is induced in all the animals by laser photocoagulation in several places of the retina (4 to 5 laser impacts per eye).

### Test Results

Fourteen days after lesion (D17), the vascular leakage of the neovascularization is evaluated by fluorescent angiography and attribution of a score as a function of the severity of the vascular leakage according to the following table.

### Observation Grade

No hyperfluorescence 0
Slight hyperfluorescence without increase in intensity or size 1
Hyperfluorescence increasing in intensity in the late phase without increase in size (moderate leakage) 2
Hyperfluorescence with early phase leakage with increase in size and intensity in the late phase (severe leakage) 3

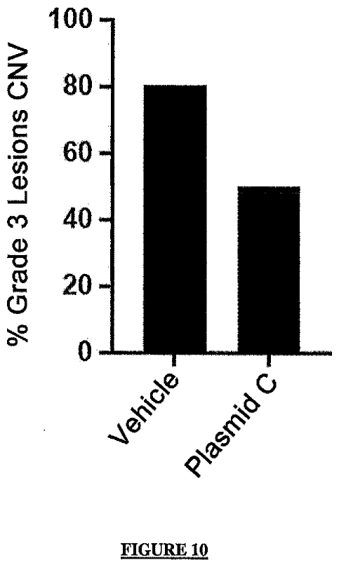

The results obtained are shown in FIG. 10 and represent in particular the percentage impact with severe leakage (grade 3) as a function of the treatments.

On examining this figure, it appears that the plasmid according to the invention gives a 38% reduction in the number of impacts showing severe neovascular leakage compared to the animals that received the vehicle.

Sequence listing

SEQ ID NO: 1: nucleotide sequence coding for aflibercept
agtgatacaggtagacctttcgtagagatgtacagtgaaatccccgaaattatacacatgactgaaggaagggagctcgtcattccctgc
cgggttacgtcacctaacatcactgttactttaaaaaagtttccacttgacactttgatccctgatggaaaacgcataatctgggacagtag
aaagggcttcatcatatcaaatgcaacgtacaaagaaatagggcttctgacctgtgaagcaacagtcaatgggcatttgtataagacaaa
ctatctcacacatcgacaaaccaatacaatcatagatgtcgttctgagtccgictcatggaattgaactatctgttggagaaaagcttgtctt
aaattgtacagcaagaactgaactaaatgtggggattgacttcaactgggaataccttcttcgaagcatcagcataagaaacttgtaaac
cgagacctaaaaacccagtctgggagtgagatgaagaaatttttgagcaccttaactatagatggtgtaacccggagtgaccaaggatt
gtacacctgtgcagcatccagtgggctgatgaccaagaagaacagcacatttgtcagggtccatgaaaaagacaaaactcacacatgc
ccaccgtgcccagcacctgaactcctggggggaccgtcagtcttcctcttccccccaaaacccaaggacaccctcatgatctcccgga
cccctgaggtcacatgcgtggtggtggacgtgagccacgaagaccctgaggtcaagttcaactggtacgtggacggcgtggaggtg
cataatgccaagacaaagccgcgggaggagcagtacaacagcacgtaccgtgtggtcagcgtcctcaccgtcctgcaccaggactg
gctgaatggcaaggagtacaagtgcaaggtctccaacaaagccctcccagcccccatcgagaaaaccatctccaaagccaaagggc
agccccgagaaccacaggtgtacaccctgcccccatcccgggaggagatgaccaagaaccaggtcagcctgacctgcctggtcaaa
ggcttctatcccagcgacatcgccgtggagtgggagagcaatgggcagccggagaacaactacaagaccacgcctcccgtgctgga
ctccgacggctccttcttcctctatagcaagctcaccgtggacaagagcaggtggcagcaggggaacgtcttctcatgctccgtgatgc
atgaggctctgcacaaccactacacgcagaagagcctctccctgtccccgggtaaa
SEQ ID NO: 2: nucleotide sequence coding for aflibercept
cagcgacaccggcagacccttcgtggaaatgtacagcgagatccccgagatcatccacatgaccgagggccgcgagctggtgatcc
cttgcagagtgaccagccccaacatcaccgtgacactgaagaagttccctctggacacactgatccccgacggcaagaggatcatctg
ggacagcagaaagggcttcatcatcagcaacgccacatacaaagagatcggactgctgacatgcgaggccaccgtgaacggccatc
tgtacaagaccaactatctgacccaccgccagaccaacaccatcatcgacgtggtgctgagccccagccacggcatcgagctgagcg
tgggcgagaagctggtgctgaactgcaccgccagaaccgagctgaatgtgggcatcgacttcaactgggagtaccccagctccaag
caccagcacaagaaactggtgaaccgggatctgaaaacccagagcggcagcgagatgaagaagtttctgagcacactgaccatcga
cggcgtgaccagaagcgaccaaggactgtacacatgcgccgccagcagcggactgatgaccaagaagaacagcacattcgtccgg
gtgcacgagaaggacaagacccacacatgcccaccatgcccagccccagagctgctgggaggcccctccgtgtttctgttccctcca
aagcccaaggacactctgatgatcagcagaacccccgaagtgacatgcgtggtggtggacgtgtcccacgaggacccagaagtgaa
gttcaattggtacgtggacggcgtggaagtgcacaacgccaagaccaagcccagagaggaacagtacaacagcacatacagagtg
gtgtccgtgctgaccgtgctgcaccaagactggctgaacggcaaagagtacaagtgcaaagtctccaacaaggctctgccagccccc
atcgaaaagaccatcagcaaggccaagggccagcctcgcgagccccaagtgtacacactgcctccaagccgggacgagctgacca
agaatcaagtgtctctgacatgtctggtgaaaggcttctaccccagcgatatcgccgtggaatgggagagcaacggccagcccgaga
acaactacaagaccacccctccgtgctggacagcgacggcagcttctttctgtactccaaactgaccgtggacaagagcagatggca
gcaaggcaacgtgttcagctgcagcgtgatgcacgaggctctgcacaaccactacacccagaagtctctgtctctgagccccggcaa
gtga
SEQ ID NO: 3: peptide sequence of aflibercept
SDTGRPFVEMYSEIPEIIHMTEGRELVIPCRVTSPNITVTLKKFPLDTLIPDGKRIIWDSR
KGFIISNATYKEIGLLTCEATVNGHLYKTNYLTHRQTNTIIDVVLSPSHGIELSVGEKL
VLNCTARTELNVGIDFNWEYPSSKHQHKKLVNRDLKTQSGSEMKKFLSTLTIDGVTR
SDQGLYTCAASSGLMTKKNSTFVRVHEKDKTHTCPPCPAPELLGGPSVFLFPPKPKDT
LMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVDKSRWQQG
NVFSCSVMHEALHNHYTQKSLSLSPGK
SEQ ID NO: 4: peptide sequence of the TPA signal peptide
MDAMKRGLCCVLLLCGAVFVSPS
SEQ ID NO: 5: nucleotide sequence coding for the TPA signal peptide
atggatgcaatgaagagagggctctgctgtgtgctgctgctgtgtggagcagtcttcgtttcgcccag
SEQ ID NO: 6: Nucleotide sequence coding for decorin
ggaccgtttcaacagagaggcttatttgactttatgctagaagatgaggcttctgggataggcccagaagttcctgatgaccgcgacttc
gagccctccctaggcccagtgtgccccttccgctgtcaatgccatcttcgagtggtccagtgttctgatttgggtctggacaaagtgccaa
aggatcttccccctgacacaactctgctagacctgcaaaacaacaaaataaccgaaatcaaagatggagactttaagaacctgaagaa
ccttcacgcattgattcttgtcaacaataaaattagcaaagttagtcctggagcatttacacctttggtgaagttggaacgactttatctgtcc
aagaatcagctgaaggaattgccagaaaaaatgcccaaaactcttcaggagctgcgtgcccatgagaatgagatcaccaaagtgcga
aaagttactttcaatggactgaaccagatgattgtcatagaactgggcaccaatccgctgaagagctcaggaattgaaaatggggctttc
cagggaatgaagaagctctcctacatccgcattgctgataccaatatcaccagcattcctcaaggtcttcctccttcccttacggaattaca
tcttgatggcaacaaaatcagcagagttgatgcagctagcctgaaaggactgaataatttggctaagttgggattgagtttcaacagcatc
tctgctgttgacaatggctctctggccaacacgcctcatctgagggagcttcacttggacaacaacaagcttaccagagtacctggtggg
ctggcagagcataagtacatccaggttgtctaccttcataacaacaatatctctgtagttggatcaagtgacttctgcccacctggacaca
acaccaaaaaggcttcttattcgggtgtgagtcttttcagcaacccggtccagtactgggagatacagccatccaccttcagatgtgtcta
cgtgcgctctgccattcaactcggaaactataagtaa
SEQ ID NO: 7: Nucleotide sequence coding for decorin
ggaccgtttcaacagagaggcttatttgactttatgctagaagatgaggccagcggcatcggccccgaagtgcccgatgatagagattt
cgagccctctctgggccccgtgtgtcctttcagatgccagtgtcatctgagagtggtgcagtgcagcgatctgggcctcgacaaagtgc
ctaaggatctgcctccagacaccacactgctggatctgcagaacaacaagatcaccgagatcaaggacggcgactttaagaatctgaa
gaatctccacgctctgatcctcgtgaacaacaaaatctccaaagtgtctcccggcgctttcacccctctggtcaagctggaacggctgtat
ctgagcaagaaccagctgaaagaactgcccgagaagatgcccaagacactgcaagagctgagagcccacgagaacgagatcacc
aaagtgcggaaagtgacattcaacgggctgaaccagatgatcgtgatcgagctgggcaccaatcctctgaagtcctccggaatcgag
aacggcgccttccaaggcatgaagaagctgagctacatccggatcgccgacaccaacatcaccagcattcctcaagggctgcctcca
tctctgaccgagctgcatctggacggcaacaagatttccagagtggacgccgcctctctgaagggactgaacaatctggccaaactgg
gactgagcttcaacagcatcagcgccgtggacaacggctctctggccaacacaccacatctgcgggaactccatctggataacaaca
agctgaccagagttcccggcggactggccgagcacaagtacatccaagtggtgtatctccacaacaacaatatcagcgtcgtgggca
gcagcgatttctgccctccgggacacaataccaagaaggccagctacagcggagtgtctctgttcagcaatcccgtgcagtactggga
gatccagcctagcacattcagatgcgtgtacgtgcggagcgccatccagctgggcaactacaagtgatga
SEQ ID NO: 8: Peptide sequence of decorin
GPFQQRGLFDFMLEDEASGIGPEVPDDRDFEPSLGPVCPFRCQCHLRVVQCSDLGLD
KVPKDLPPDTTLLDLQNNKITEIKDGDFKNLKNLHALILVNNKISKVSPGAFTPLVKL
ERLYLSKNQLKELPEKMPKTLQELRAHENEITKVRKVTFNGLNQMIVIELGTNPLKSS
GIENGAFQGMKKLSYIRIADTNITSIPQGLPPSLTELHLDGNKISRVDAASLKGLNNLA
KLGLSFNSISAVDNGSLANTPHLRELHLDNNKLTRVPGGLAEHKYIQVVYLHNNNIS
VVGSSDFCPPGHNTKKASYSGVSLFSNPVQYWEIQPSTFRCVYVRSAIQLGNYK
SEQ ID NO: 9: Nucleotide sequence coding for decorin
ggaccgtttcaacagagaggcttatttgactttatgctagaagatgaggcctctggaatcggacctgaggtgcccgacgacagagactt
cgaaccttctctgggccctgtgtgccccttcagatgccagtgtcatctgagagtggtgcagtgcagcgacctgggccttgataaggtgc
ccaaggacctgcctcctgacaccacactgctggacctgcagaacaacaagatcaccgagatcaaggacggcgacttcaagaacctg
aagaatctgcacgccctgatcctggtcaacaacaaaatcagcaaggtgtcccctggcgccttcacacctctggtcaagctggaaagact
gtacctgagcaagaaccagctgaaagaactgcccgagaagatgcccaagacactgcaagagctgcgggcccacgagaacgagatc
accaaagtgcggaaagtgaccttcaacggcctgaaccagatgatcgtgatcgagctgggcaccaatcctctgaagtccagcggcattg
agaacggcgccttccagggcatgaagaagctgagctacatccggatcgccgacaccaacatcaccagcattcctcagggcctgcctc
caagcctgacagagctgcatctggacggcaacaagattagcagagtggacgccgcctctctgaagggcctgaacaatctggccaaa
ctgggcctgagcttcaacagcatcagcgccgtggataacggcagcctggccaacacacctcacctgagggaactgcacctggataac
aacaagctgaccagagtgcctggcggactggccgagcacaagtacatccaggtggtgtatctccacaacaacaacatctccgtcgtg
ggcagcagcgacttctgtcctcctggccacaataccaagaaggccagctactctggcgtgtccctgttcagcaaccccgtgcagtactg
ggagatccagcctagcacctttagatgcgtgtacgtgcggagcgccatccagctgggcaactacaaatga
SEQ ID NO: 10: Nucleotide sequence coding for decorin
ggaccgtttcaacagagaggcttatttgactttatgctagaagacgaggctagcggaattggacctgaagtgcccgacgaccgcgatttt
gaaccatcactgggacctgtctgcccctttagatgtcagtgccacctgagggtggtgcagtgttctgacctgggcctggataaggtgcc
aaaggacctgccccctgataccacactgctggacctgcagaacaataagatcaccgagatcaaggacggcgatttcaagaatctgaa
gaacctgcacgccctgatcctggtgaacaataagatctctaaggtgagcccaggcgcctttacccccctggtgaagctggagagactg
tacctgagcaagaatcagctgaaggagctgcccgagaagatgcctaagacactgcaggagctgcgggcccacgagaacgagatca
ccaaggtgagaaaggtgacattcaatggcctgaaccagatgatcgtgatcgagctgggcaccaatcccctgaagagctccggcatcg
agaacggcgcctttcagggcatgaagaagctgtcctatatccggatcgccgacaccaatatcacatctatccctcagggcctgccaccc
agcctgacagagctgcacctggacggcaacaagatcagcagagtggatgccgcctccctgaagggcctgaacaatctggccaagct
gggcctgtccttcaactccatctctgccgtggacaatggctctctggccaacacccctcacctgagggagctgcacctggataacaata
agctgacacgcgtgccaggcggcctggcagagcacaagtacatccaggtggtgtatctgcacaacaataacatctccgtggtgggct
ctagcgatttctgccctccaggccacaatacaaagaaggccagctactccggcgtgtccctgttttctaaccctgtgcagtattgggagat
ccagccctctacttttcggtgcgtctatgtcaggtccgccattcagctggggaactacaaataa
SEQ ID NO: 11: Nucleotide sequence coding for decorin
ggaccgtttcaacagagaggcttatttgactttatgctagaagacgaggccagcggcatcggccccgaggtgcccgacgaccgcgac
ttcgagcccagcctgggccccgtgtgccccttccgctgccagtgccacctgcgcgtggtgcagtgcagcgacctgggcctggacaag
gtgcccaaggacctgccccccgacaccaccctgctggacctgcagaacaacaagatcaccgagatcaaggacggcgacttcaaga
acctgaagaacctgcacgccctgatcctggtgaacaacaagatcagcaaggtgagccccggcgccttcacccccctggtgaagctgg
agcgcctgtacctgagcaagaaccagctgaaggagctgcccgagaagatgcccaagaccctgcaggagctgcgcgcccacgaga
acgagatcaccaaggtgcgcaaggtgaccttcaacggcctgaaccagatgatcgtgatcgagctgggcaccaaccccctgaagagc
agcggcatcgagaacggcgccttccagggcatgaagaagctgagctacatccgcatcgccgacaccaacatcaccagcatccccca
gggcctgccccccagcctgaccgagctgcacctggacggcaacaagatcagccgcgtggacgccgccagcctgaagggcctgaa
caacctggccaagctgggcctgagcttcaacagcatcagcgccgtggacaacggcagcctggccaacaccccccacctgcgcgag
ctgcacctggacaacaacaagctgacccgcgtgcccggcggcctggccgagcacaagtacatccaggtggtgtacctgcacaacaa
caacatcagcgtggtgggcagcagcgacttctgcccccccggccacaacaccaagaaggccagctacagcggcgtgagcctgttca
gcaaccccgtgcagtactgggagatccagcccagcaccttccgctgcgtgtacgtgcgcagcgccatccagctgggcaactacaagt
aa
SEQ ID NO: 12: Nucleotide sequence coding for decorin
ggaccgtttcaacagagaggcttatttgactttatgctagaagatgaggcgagtggcattggacctgaagtacccgatgatagagacttt
gaaccatcattgggcccagtttgcccttttaggtgtcagtgccacctccgggtagttcaatgcagcgatttgggactcgataaagtaccga
aagacttgccaccggacacaacattgctcgatcttcaaaacaacaagatcactgaaataaaggatggagactttaaaaatctgaagaatt
tgcacgccctcatcctggtcaacaacaagatcagcaaggtgtcccctggagcattcacgcccctcgtaaagttggaacgcctctacctg
tctaagaaccagttgaaagaactgcccgagaagatgcctaaaactctgcaagagcttagagctcatgaaaatgaaattaccaaggttcg
gaaggtaacctttaacggtcttaaccagatgatagtcattgagttgggcacgaacccattgaaatcttctggcatagaaaacggggctttc
caggggatgaaaaaactctcatatatccgcatcgcggataccaacatcacatctatacctcaaggtttgcccccgagtttgaccgagctt
cacctggatggcaacaagataagccgggtcgacgctgcctcactcaaagggctcaataatctggcgaaactggggttgagtttcaattc
aatatctgctgtcgacaacggctcacttgcgaacacaccccatcttagggaacttcatctggacaacaacaagttgacacgggttcctgg
gggactcgctgaacataaatatatacaggtcgtttatctccataataataatatcagcgttgtaggctcatctgacttctgccctccaggcca
taatacaaagaaagcgtcatacagtggcgtcagtttgttctctaacccggttcagtattgggagattcaaccgtccacttttcggtgcgttta
cgtgaggagtgcgattcagctgggtaactataagtaa
SEQ ID NO: 13: Peptide sequence of the native signal peptide of decorin
MKATIILLLLAQVSWA
SEQ ID NO: 14: Nucleotide sequence coding for the native signal peptide of decorin
atgaaggccactatcatcctccttctgcttgcacaagtttcctgggct
SEQ ID NO: 15: Nucleotide sequence coding for transferrin
gtccctgataaaactgtgagatggtgtgcagtgtcggagcatgaggccactaagtgccagagtttccgcgaccatatgaaaagcgtcat
tccatccgatggtcccagtgttgcttgtgtgaagaaagcctcctaccttgattgcatcagggccattgcggcaaacgaagcggatgctgt
gacactggatgcaggtttggtgtatgatgcttacctggctcccaataacctgaagcctgtggtggcagagttctatgggtcaaaagagga
tccacagactttctattatgctgttgctgtggtgaagaaggatagtggcttccagatgaaccagcttcgaggcaagaagtcctgccacac
gggtctaggcaggtccgctgggtggaacatccccataggcttactttactgtgacttacctgagccacgtaaacctcttgagaaagcagt
ggccaatttcttctcgggcagctgtgccccttgtgcggatgggacggacttcccccagctgtgtcaactgtgtccagggtgtggctgctc
cacccttaaccaatacttcggctactcgggagccttcaagtgtctgaaggatggtgctggggatgtggcctttgtcaagcactcgactata
tttgagaacttggcaaacaaggctgacagggaccagtatgagctgctttgcctggacaacacccggaagccggtagatgaatacaag
gactgccacttggcccaggtcccttctcataccgtcgtggcccgaagtatgggcggcaaggaggacttgatctgggagcttctcaacc
aggcccaggaacattttggcaaagacaaatcaaaagaattccaactattcagctctcctcatgggaaggacctgctgtttaaggactctg
cccacgggtttttaaaagtcccccccaggatggatgccaagatgtacctgggctatgagtatgtcactgccatccggaatctacgggaa
ggcacatgcccagaagccccaacagatgaatgcaagcctgtgaagtggtgtgcgctgagccaccacgagaggctcaagtgtgatga
gtggagtgttaacagtgtagggaaaatagagtgtgtatcagcagagaccaccgaagactgcatcgccaagatcatgaatggagaagct
gatgccatgagcttggatggagggtttgtctacatagcgggcaagtgtggtctggtgcctgtcttggcagaaaactacaataagagcga
taattgtgaggatacaccagaggcagggtattttgctatagcagtggtgaagaaatcagcttctgacctcacctgggacaatctgaaagg
caagaagtcctgccatacggcagttggcagaaccgctggctggaacatccccatgggcctgctctacaataagatcaaccactgcaga
tttgatgaatttttcagtgaaggttgtgcccctgggtctaagaaagactccagtctctgtaagctgtgtatgggctcaggcctaaacctgtgt
gaacccaacaacaaagagggatactacggctacacaggcgctttcaggtgtctggttgagaagggagatgtggcctttgtgaaacacc
agactgtcccacagaacactgggggaaaaaaccctgatccatgggctaagaatctgaatgaaaaagactatgagttgctgtgccttgat
ggtaccaggaaacctgtggaggagtatgcgaactgccacctggccagagccccgaatcacgctgtggtcacacggaaagataagga
agcttgcgtccacaagatattacgtcaacagcagcacctatttggaagcaacgtaactgactgctcgggcaacttttgtttgttccggtcg
gaaaccaaggaccttctgttcagagatgacacagtatgtttggccaaacttcatgacagaaacacatatgaaaaatacttaggagaaga
atatgtcaaggctgttggtaacctgagaaaatgctccacctcatcactcctggaagcctgcactttccgtagaccttaa
SEQ ID NO: 16: Nucleotide sequence coding for transferrin
gtgccagataagacagttcgttggtgcgccgtgtctgagcacgaggccacaaagtgccagagcttccgggaccacatgaagtctgtg
atccctagcgacggcccttccgtggcttgtgtgaagaaggccagctatctggactgcatcagagccattgccgccaacgaagccgatg
ccgttacactggatgccggactggtgtacgatgcctatctggccccaaacaatctgaagcccgtggtcgccgagttctacggctctaaa
gaggaccctcagacattctactacgccgtggccgtggtcaagaaggacagcggctttcagatgaaccagctgcggggcaagaagtct
tgtcacaccggacttggaagaagcgccggctggaatatccccatcggactgctgtactgcgatctgcccgagcctagaaagcctctgg
aaaaggccgtggccaacttcttctctggctcttgtgccccttgcgccgatggcacagattttccacagctctgtcagctgtgtcccggctg
tggctgtagcacactgaaccagtactttggctacagcggcgccttcaagtgtctgaaagatggtgctggcgacgtggccttcgtgaagc
acagcacaatcttcgagaatctggccaacaaggccgaccgggatcagtacgaactgctgtgcctcgacaacaccagaaagccagtg
gacgagtacaaggactgccatctggctcaagtgcctagccacacagtggttgccagatccatgggggcaaagaggatctgatctgg
gagctgctgaatcaagcccaagagcacttcggcaaggacaagagcaaagagttccagctgttcagcagccctcacggcaaggatct
gtgttcaaggatagcgcccacggatttctgaaagtgcctcctcggatggacgccaagatgtatctgggctacgagtacgtgaccgcc
atccggaatctgagagaaggcacatgcccagaggctcccaccgatgagtgtaaaccagtgaagtggtgcgctctgtctcaccacgag
agactgaagtgtgacgagtggtccgtgaacagcgtgggcaagattgagtgtgtgtccgccgagacaaccgaggactgtatcgccaag
atcatgaacggcgaggccgacgctatgtctctggatggcggatttgtgtacattgccggaaagtgtggactggtgccagtgctggccg
agaactacaacaagagcgacaactgcgaggataccccagaggccggatattttgccgtggcagtcgtgaagaagtccgccagcgat
ctgacatgggacaatctcaagggcaagaaaagctgccacaccgccgtgggaagaacagccggatggaacattcctatggggctgct
gtacaacaaaatcaaccactgccgcttcgacgagttcttcagcgaaggatgtgctcccggcagcaagaaagacagctctctgtgcaag
ctgtgcatgggcagcggactgaatctgtgcgagcccaacaacaaagagggctactacggctacaccggggcctttagatgtctggttg
agaagggcgacgttgcatttgtgaaacaccagaccgtgcctcagaacaccggcggcaagaatcccgatccttgggccaagaatctga
acgagaaggactatgagctgctctgtctggacggcacccggaaaccagtggaagaatacgccaactgtcatctggcaagagcccca
aatcacgccgtcgtgaccagaaaggacaaagaggcttgcgtccacaagattctgcggcagcagcagcatctgttcggcagcaatgtg
accgactgcagcggcaacttctgtctgttcagaagcgagacaaaggatctcctcttccgcgacgataccgtgtgtctcgccaagctgca
cgaccggaacacatacgagaagtatctgggagaagagtatgtgaaggctgtgggcaatctgcggaagtgcagcacatcttctctgctc
gaggcttgcacatttcggcggccttgatga
SEQ ID NO: 17: Peptide sequence of human transferrin
VPDKTVRWCAVSEHEATKCQSFRDHMKSVIPSDGPSVACVKKASYLDCIRAIAANEA
DAVTLDAGLVYDAYLAPNNLKPVVAEFYGSKEDPQTFYYAVAVVKKDSGFQMNQL
RGKKSCHTGLGRSAGWNIPIGLLYCDLPEPRKPLEKAVANFFSGSCAPCADGTDFPQL
CQLCPGCGCSTLNQYFGYSGAFKCLKDGAGDVAFVKHSTIFENLANKADRDQYELL
CLDNTRKPVDEYKDCHLAQVPSHTVVARSMGGKEDLIWELLNQAQEHFGKDKSKEF
QLFSSPHGKDLLFKDSAHGFLKVPPRMDAKMYLGYEYVTAIRNLREGTCPEAPTDEC
KPVKWCALSHHERLKCDEWSVNSVGKIECVSAETTEDCIAKIMNGEADAMSLDGGF
VYIAGKCGLVPVLAENYNKSDNCEDTPEAGYFAVAVVKKSASDLTWDNLKGKKSC
HTAVGRTAGWNIPMGLLYNKINHCRFDEFFSEGCAPGSKKDSSLCKLCMGSGLNLCE
PNNKEGYYGYTGAFRCLVEKGDVAFVKHQTVPQNTGGKNPDPWAKNLNEKDYELL
CLDGTRKPVEEYANCHLARAPNHAVVTRKDKEACVHKILRQQQHLFGSNVTDCSGN
FCLFRSETKDLLFRDDTVCLAKLHDRNTYEKYLGEEYVKAVGNLRKCSTSSLLEACT
FRRP
SEQ ID NO: 18: Peptide sequence of the native signal peptide of human transferrin
MRLAVGALLVCAVLGLCLA
SEQ ID NO: 19: Nucleotide sequence coding for the native signal peptide of transferrin
atgaggctcgccgtgggagccctgctggtctgcgccgtcctggggctgtgtctggct
SEQ ID NO: 20: Nucleotide sequence coding for the signal peptide of transferrin
atgagactggctgtgggagcactgcttgtgtgtgctgttctgggactgtgtctggcc
SEQ ID NO: 21: nucleotide sequence coding for a fusion protein comprising the extracellular
domain of the human receptor p55 to TNF alpha coupled via a hinge to the constant fragment
of human immunoglobulin IgG1 (Peppel et al., J Exp Med, 174: 1483-1489-Murphy et al.,
Arch Ophthalmol, 22: 845-851)
actggtccctcacctaggggacagggagaagagagatagtgtgtgtccccaaggaaaatatatccaccctcaaaataattcgatttgct
gtaccaagtgccacaaaggaacctacttgtacaatgactgtccaggcccggggcaggatacggactgcagggagtgtgagagcggc
tccttcaccgcttcagaaaaccacctcagacactgcctcagctgctccaaatgccgaaaggaaatgggtcaggtggagatctcttcttgc
acagtggaccgggacaccgtgtgtggctgcaggaagaaccagtaccggcattattggagtgaaaaccttttccagtgcttcaattgcag
cctctgcctcaatgggaccgtgcacctctcctgccaggagaaacagaacaccgtgtgcacctgccatgcaggtttctttctaagagaaa
acgagtgtgtctcctgtagtaactgtaagaaaagcctggagtgcacgaagttgtgcctaccccagattgagaatgttaagggcactgag
gactcaggcaccacactggttccgcgtggatccgacaaaactcacacatgcccaccgtgcccagcacctgaactcctggggggacc
gtcagtcttcctcttccccccaaaacccaaggacaccctcatgatctcccggacccctgaggtcacatgcgtggtggtggacgtgagcc
acgaagaccctgaggtcaagttcaactggtacgtggacggcgtggaggtgcataatgccaagacaaagccgcgggaggagcagta
caacagcacgtaccgtgtggtcagcgtcctcaccgtcctgcaccaggactggctgaatggcaaggagtacaagtgcaaggtctccaa
caaagccctcccagcccccatcgagaaaaccatctccaaagccaaagggcagccccgagaaccacaggtgtacaccctgcccccat
cccgggaggagatgaccaagaaccaggtcagcctgacctgcctggtcaaaggcttctatcccagcgacatcgccgtggagtgggag
agcaatgggcagccggagaacaactacaagaccacgcctcccgtgctggactccgacggctccttcttcctctacagcaagctcacc
gtggacaagagcaggtggcagcaggggaacgtcttctcatgctccgtgatgcacgaggctctgcacaaccactacacgcagaagag
cctctccctgtctccgggtaaatga
SEQ ID NO: 22: peptide sequence of the fusion protein comprising the extracellular domain
of the human receptor p55 to TNF alpha coupled via a hinge to the constant fragment of human
immunoglobulin IgG1 (Peppel et al., J Exp Med, 174: 1483-1489-Murphy et al., Arch
Ophthalmol, 22: 845-851)
LVPHLGDREKRDSVCPQGKYIHPQNNSICCTKCHKGTYLYNDCPGPGQDTDCRECES
GSFTASENHLRHCLSCSKCRKEMGQVEISSCTVDRDTVCGCRKNQYRHYWSENLFQ
CFNCSLCLNGTVHLSCQEKQNTVCTCHAGFFLRENECVSCSNCKKSLECTKLCLPQIE
NVKGTEDSGTTLVPRGSDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNG
KEYKCKVSNKALPAPIEKTISKAKGQPREPQVYTLPPSREEMTKNQVSLTCLVKGFYP
SDIAVEWESNGQPENNYKTTPPVLDSDGSFFLYSKLTVDKSRWQQGNVFSCSVMHE
ALHNHYTQKSLSLSPGK
SEQ ID NO: 23: Peptide sequence of the native signal peptide of the protein of sequence SEQ
ID NO: 22
MGLSTVPDLLLPLVLLELLVGIYPSGVIG
SEQ ID NO: 24: Nucleotide sequence coding for the signal peptide of sequence SEQ ID NO: 23
atgggcctctccaccgtgcctgacctgctgctgccgctggtgctcctggagctgttggtgggaatatacccctcaggggttattgg
SEQ ID NO: 25: nucleotide sequence coding for complement factor H
gaagattgcaatgaacttcctccaagaagaaatacagaaattctgacaggttcctggtctgaccaaacatatccagaaggcacccaggc
tatctataaatgccgccctggatatagatctcttggaaatataataatggtatgcaggaagggagaatgggttgctcttaatccattaagga
aatgtcagaaaaggccctgtggacatcctggagatactccttttggtacttttacccttacaggaggaaatgtgtttgaatatggtgtaaaa
gctgtgtatacatgtaatgaggggtatcaattgctaggtgagattaattaccgtgaatgtgacacagatggatggaccaatgatattcctat
atgtgaagttgtgaagtgtttaccagtgacagcaccagagaatggaaaaattgtcagtagtgcaatggaaccagatcgggaataccattt
tggacaagcagtacggtttgtatgtaactcaggctacaagattgaaggagatgaagaaatgcattgttcagacgatggtttttggagtaaa
gagaaaccaaagtgtgtggaaatttcatgcaaatccccagatgttataaatggatctcctatatctcagaagattatttataaggagaatga
acgatttcaatataaatgtaacatgggttatgaatacagtgaaagaggagatgctgtatgcactgaatctggatggcgtccgttgccttcat
gtgaagaaaaatcatgtgataatccttatattccaaatggtgactactcacctttaaggattaaacacagaactggagatgaaatcacgtac
cagtgtagaaatggtttttatcctgcaacccggggaaatacagcaaaatgcacaagtactggctggatacctgctccgagatgtaccttg
aaaccttgtgattatccagacattaaacatggaggtctatatcatgagaatatgcgtagaccatactttccagtagctgtaggaaaatattac
tcctattactgtgatgaacattttgagactccgtcaggaagttactgggatcacattcattgcacacaagatggatggtcgccagcagtac
catgcctcagaaaatgttattttccttatttggaaaatggatataatcaaaattatggaagaaagtttgtacagggtaaatctatagacgttgc
ctgccatcctggctacgctcttccaaaagcgcagaccacagttacatgtatggagaatggctggtctcctactcccagatgcatccgtgt
caaaacatgttccaaatcaagtatagatattgagaatgggtttatttctgaatctcagtatacatatgccttaaaagaaaaagcgaaatatca
atgcaaactaggatatgtaacagcagatggtgaaacatcaggatcaattacatgtgggaaagatggatggtcagctcaacccacgtgc
attaaatcttgtgatatcccagtatttatgaatgccagaactaaaaatgacttcacatggtttaagctgaatgacacattggactatgaatgc
catgatggttatgaaagcaatactggaagcaccactggttccatagtgtgtggttacaatggttggtctgatttacccatatgttatgaaaga
gaatgcgaacttcctaaaatagatgtacacttagttcctgatcgcaagaaagaccagtataaagttggagaggtgttgaaattctcctgca
aaccaggatttacaatagttggacctaattccgttcagtgctaccactttggattgtctcctgacctcccaatatgtaaagagcaagtacaat
catgtggtccacctcctgaactcctcaatgggaatgttaaggaaaaaacgaaagaagaatatggacacagtgaagtggtggaatattatt
gcaatcctagatttctaatgaagggacctaataaaattcaatgtgttgatggagagtggacaactttaccagtgtgtattgtggaggagagt
acctgtggagatatacctgaacttgaacatggctgggcccagctttcttcccctccttattactatggagattcagtggaattcaattgctca
gaatcatttacaatgattggacacagatcaattacgtgtattcatggagtatggacccaacttccccagtgtgtggcaatagataaacttaa
gaagtgcaaatcatcaaatttaattatacttgaggaacatttaaaaaacaagaaggaattcgatcataattctaacataaggtacagatgta
gaggaaaagaaggatggatacacacagtctgcataaatggaagatgggatccagaagtgaactgctcaatggcacaaatacaattatg
cccacctccacctcagattcccaattctcacaatatgacaaccacactgaattatcgggatggagaaaaagtatctgttctttgccaagaa
aattatctaattcaggaaggagaagaaattacatgcaaagatggaagatggcagtcaataccactctgtgttgaaaaaattccatgttcac
aaccacctcagatagaacacggaaccattaattcatccaggtcttcacaagaaagttatgcacatgggactaaattgagttatacttgtga
gggtggtttcaggatatctgaagaaaatgaaacaacatgctacatgggaaaatggagttctccacctcagtgtgaaggccttccttgtaa
atctccacctgagatttctcatggtgttgtagctcacatgtcagacagttatcagtatggagaagaagttacgtacaaatgttttgaaggtttt
ggaattgatgggcctgcaattgcaaaatgcttaggagaaaaatggtctcaccctccatcatgcataaaaacagattgtctcagtttaccta
gctttgaaaatgccatacccatgggagagaagaaggatgtgtataaggcgggtgagcaagtgacttacacttgtgcaacatattacaaa
atggatggagccagtaatgtaacatgcattaatagcagatggacaggaaggccaacatgcagagacacctcctgtgtgaatccgccca
cagtacaaaatgcttatatagtgtcgagacagatgagtaaatatccatctggtgagagagtacgttatcaatgtaggagcccttatgaaat
gtttggggatgaagaagtgatgtgtttaaatggaaactggacggaaccacctcaatgcaaagattctacaggaaaatgtgggccccctc
cacctattgacaatggggacattacttcattcccgttgtcagtatatgctccagcttcatcagttgagtaccaatgccagaacttgtatcaac
ttgagggtaacaagcgaataacatgtagaaatggacaatggtcagaaccaccaaaatgcttacatccgtgtgtaatatcccgagaaatta
tggaaaattataacatagcattaaggtggacagccaaacagaagctttattcgagaacaggtgaatcagttgaatttgtgtgtaaacggg
gatatcgtctttcatcacgttctcacacattgcgaacaacatgttgggatgggaaactggagtatccaacttgtgcaaaaagatag
SEQ ID NO: 26: peptide sequence of complement factor H
EDCNELPPRRNTEILTGSWSDQTYPEGTQAIYKCRPGYRSLGNIIMVCRKGEWVALNP
LRKCQKRPCGHPGDTPFGTFTLTGGNVFEYGVKAVYTCNEGYQLLGEINYRECDTD
GWTNDIPICEVVKCLPVTAPENGKIVSSAMEPDREYHFGQAVRFVCNSGYKIEGDEE
MHCSDDGFWSKEKPKCVEISCKSPDVINGSPISQKIIYKENERFQYKCNMGYEYSERG
DAVCTESGWRPLPSCEEKSCDNPYIPNGDYSPLRIKHRTGDEITYQCRNGFYPATRGN
TAKCTSTGWIPAPRCTLKPCDYPDIKHGGLYHENMRRPYFPVAVGKYYSYYCDEHFE
TPSGSYWDHIHCTQDGWSPAVPCLRKCYFPYLENGYNQNYGRKFVQGKSIDVACHP
GYALPKAQTTVTCMENGWSPTPRCIRVKTCSKSSIDIENGFISESQYTYALKEKAKYQ
CKLGYVTADGETSGSITCGKDGWSAQPTCIKSCDIPVFMNARTKNDFTWFKLNDTLD
YECHDGYESNTGSTTGSIVCGYNGWSDLPICYERECELPKIDVHLVPDRKKDQYKVG
EVLKFSCKPGFTIVGPNSVQCYHFGLSPDLPICKEQVQSCGPPPELLNGNVKEKTKEE
YGHSEVVEYYCNPRFLMKGPNKIQCVDGEWTTLPVCIVEESTCGDIPELEHGWAQLS
SPPYYYGDSVEFNCSESFTMIGHRSITCIHGVWTQLPQCVAIDKLKKCKSSNLIILEEH
LKNKKEFDHNSNIRYRCRGKEGWIHTVCINGRWDPEVNCSMAQIQLCPPPPQIPNSH
NMTTTLNYRDGEKVSVLCQENYLIQEGEEITCKDGRWQSIPLCVEKIPCSQPPQIEHG
TINSSRSSQESYAHGTKLSYTCEGGFRISEENETTCYMGKWSSPPQCEGLPCKSPPEIS
HGVVAHMSDSYQYGEEVTYKCFEGFGIDGPALAKCLGEKWSHPPSCIKTDCLSLPSFE
NAIPMGEKKDVYKAGEQVTYTCATYYKMDGASNVTCINSRWTGRPTCRDTSCVNPP
TVQNAYIVSRQMSKYPSGERVRYQCRSPYEMFGDEEVMCLNGNWTEPPQCKDSTGK
CGPPPPIDNGDITSFPLSVYAPASSVEYQCONLYQLEGNKRITCRNGQWSEPPKCLHP
CVISREIMENYNIALRWTAKQKLYSRTGESVEFVCKRGYRLSSRSHTLRTTCWDGKL
EYPTCAKR
SEQ ID NO: 27: Peptide sequence of the native signal peptide of factor H
MRLLAKIICLMLWAICVA
SEQ ID NO: 28: Nucleotide sequence coding for the native signal peptide of factor H
atgagacttctagcaaagattatttgccttatgttatgggctatttgtgtagca
SEQ ID NO: 29: peptide sequence of the signal peptide of HTLV-1 Env
MGKFLATLILFFQFCPLIFG
SEQ ID NO: 30: nucleotide sequence coding for the signal peptide of HTLV-1 Env
atgggtaagittctcgccactttgattttattcttccagttctgccccctcatcttcggt
SEQ ID NO: 31: sequence of the origin of replication gamma of the E. coli R6K plasmid
gatcagcagttcaacctgttgatagtatgtactaagctctcatgtttaatgtactaagctctcatgtttaatgaactaaaccctcatggctaatg
tactaagctctcatggctaatgtactaagctctcatgtttcacgtactaagctctcatgtttgaacaataaaattaatataaatcagcaacttaa
atagcctctaaggttttaagttttataagaaaaaaaagaatatataaggcttttaaagcttttaaggtttaatggttgtggacaacaagcc

What is claimed is:

1. A method for the treatment of an ocular pathology comprising the administration to an individual in need thereof a DNA construct,
said DNA construct being intended for the nonviral transfer of nucleic acids into the muscle cells of the ocular sphere of a patient with said ocular pathology;
said DNA construct being characterized in that it comprises:
(a) a bacterial or prokaryotic origin of replication, in particular bacterial,
(b) one or more sequences promoting the expression of DNA in the patient's ocular sphere,
(c) a first nucleotide sequence coding for:
a first therapeutic protein, and
a signal peptide allowing secretion of this first therapeutic protein,
this signal peptide being contiguous with the sequence of the first therapeutic protein, at the N-terminal of said first therapeutic protein,
(d) a promoter allowing expression of this first therapeutic protein in the patient's ocular sphere;
(e) a polyadenylation sequence at 3′ of the first nucleotide sequence;
(f) a second nucleotide sequence coding for:
a second therapeutic protein, different than the first therapeutic protein, and
a signal peptide allowing secretion of this second therapeutic protein,
the signal peptide being contiguous with the sequence of the second therapeutic protein, at
the N-terminal of said second therapeutic protein; and
(g) a promoter allowing expression of this second therapeutic protein in the patient's ocular sphere, and
(h) a polyadenylation sequence at 3′ of the second nucleotide sequence;
said DNA construct being administered to the patient by injection into a ciliary muscle and then electrotransfer into the cells of the ciliary muscle.

2. The method as claimed in claim 1, wherein the first therapeutic protein is a protein of the anti-VEGF type, in particular selected from the group consisting of S-Flt1, aflibercept, conbercept, brolucizumab, and in particular a protein having at least 85% sequence identity with the peptide sequence SEQ ID NO: 3, this protein more particularly being aflibercept.

3. The method as claimed in claim 1, in which the first therapeutic protein is encoded by a nucleotide sequence having at least 75% identity with the sequence SEQ ID NO: 1, and is more particularly encoded by the nucleotide sequence SEQ ID NO: 2.

4. The method as claimed in claim 1, in which:
(c) the first nucleotide sequence codes for:
a protein having at least 85% sequence identity with the sequence SEQ ID NO: 3, this protein more particularly being aflibercept; and
a signal peptide of peptide sequence SEQ ID NO: 4.

5. The method as claimed in claim 1, in which the second therapeutic protein is a protein having at least 85% sequence identity with the sequence SEQ ID NO: 8, this protein more particularly being decorin.

6. The method as claimed in claim 1, in which the second therapeutic protein is encoded by a nucleotide sequence having at least 70% sequence identity with the sequence SEQ ID NO: 6, and in particular by a sequence selected from the group consisting of the nucleotide sequences SEQ ID NO: 7, SEQ ID NO: 9, SEQ ID NO: 10, SEQ ID NO: 11 and SEQ ID NO: 12, and in particular consisting of the sequence SEQ ID NO: 7 and the sequence SEQ ID NO: 11.

7. The method as claimed in claim 1, in which:
(c) the first nucleotide sequence codes for:
a protein having at least 85% sequence identity with the sequence SEQ ID NO: 3, this protein more particularly being aflibercept; and
a signal peptide of peptide sequence SEQ ID NO: 4; and
(f) the second nucleotide sequence codes for:
a protein having at least 85% sequence identity with the sequence SEQ ID NO: 8, this protein more particularly being decorin; and
a signal peptide of peptide sequence SEQ ID NO: 13.

8. The method as claimed in claim 1, in which the origin of replication is bacterial, and is in particular an origin of replication derived from the natural plasmid R6K of Escherichia coli, in particular the origin of replication R6K gamma of the natural plasmid R6k of Escherichia coli, in particular of sequence SEQ ID NO: 31.

9. The method as claimed in claim 1, characterized in that it wherein the DNA construct is of circular shape.

10. The method as claimed in claim 1, wherein the DNA construct is naked DNA.

11. The method as claimed in claim 1, wherein the ocular pathology is a retinal degeneration, in particular a retinal degeneration selected from the group consisting of wet or dry age-related macular degeneration (ARMD); diabetic retinopathies (DR); a retinal venous occlusion, in particular a central retinal vein occlusion (CRVO) or a branch retinal vein occlusion (BRVO); a myopic choroid neovascularization (CNV); a uveitis, in particular a noninfectious uveitis; a retinitis pigmentosa and a glaucoma;
and more particularly in that the retinal degeneration is selected from the group consisting of age-related macular degeneration (ARMD), in particular the (wet) neovascular form of ARMD;
a decline of visual acuity due to diabetic macular edema (DME); a retinal venous occlusion, in particular a central retinal vein occlusion (CRVO) or a branch retinal vein occlusion (BRVO);
and a myopic choroid neovascularization (CNV).

12. A DNA construct intended for the nonviral transfer of nucleic acids into the muscle cells of a patient's ocular sphere for treating ocular pathologies, wherein the DNA construct comprises:
(a) a bacterial or prokaryotic origin of replication, in particular bacterial,
(b) one or more sequences promoting the expression of DNA in the patient's ocular sphere,
(c) a first nucleotide sequence coding for:
a first therapeutic protein, said first therapeutic protein being a protein having at least 85% sequence identity with the sequence SEQ ID NO: 3, this protein more particularly being aflibercept, and
a signal peptide allowing secretion of this first therapeutic protein, in particular a signal peptide of peptide sequence SEQ ID NO: 4,
this signal peptide being contiguous with the sequence of the first therapeutic protein, at the N-terminal of said first therapeutic protein,
(d) a promoter allowing expression of this first therapeutic protein in the patient's ocular sphere;
(e) a polyadenylation sequence at 3′ of the first nucleotide sequence;
(f) a second nucleotide sequence coding for:
a second therapeutic protein, said second therapeutic protein being a protein having at least 85% sequence identity with the sequence SEQ ID NO: 8, this protein more particularly being decorin, and
a signal peptide allowing secretion of this first therapeutic protein, in particular a signal peptide of peptide sequence SEQ ID NO: 13,
this signal peptide being contiguous with the sequence of the first therapeutic protein, at the N-terminal of said first therapeutic protein,
(g) a promoter allowing expression of this second therapeutic protein in the patient's ocular sphere; and
(h) a polyadenylation sequence at 3′ of the second nucleotide sequence.

13. The DNA construct as claimed in claim 12, in which:
(c) the first nucleotide sequence comprises:
a nucleotide sequence coding for aflibercept, and in particular a sequence having at least 75% sequence identity with the sequence SEQ ID NO: 1, and is in particular the sequence SEQ ID NO: 2; and
the nucleotide sequence SEQ ID NO: 5 coding for a signal peptide; and
(f) the second nucleotide sequence comprises:
a nucleotide sequence coding for decorin, more particularly a nucleotide sequence having at least 70% sequence identity with the sequence SEQ ID NO: 6, more particularly a sequence selected from the group consisting of the sequences SEQ ID NO: 7, SEQ ID NO: 9, SEQ ID NO: 10, SEQ ID NO: 11 and SEQ ID NO: 12, and is in particular the sequence SEQ ID NO: 7 or the sequence SEQ ID NO: 11; and
the nucleotide sequence SEQ ID NO: 14 coding for a signal peptide.
